(19) United States
(12) Patent Application Publication
(10) Pub. No.: US 2018/0036237 A1
(43) Pub. Date: Feb. 8, 2018

(54) OIL/SURFACTANT MIXTURES FOR SELF-EMULSIFICATION

(71) Applicants: GLAXOSMITHKINE BIOLOGICALSA, Rixensart (BE); NORTHEASTERN UNIVERSITY, Boston, MA (US)

(72) Inventors: Luis BRITO, Cambridge, MA (US); Derek O'HAGAN, Cambridge, MA (US); Amiji MANSOOR, Boston, MA (US); Ruchi Rudraprasad SHAH, Boston, MA (US); Manmohan SINGH, Holly Springs, MA (US)

(73) Assignees: GLAXOSMITHKLINE BIOLOGICALS, SA, Rixensart (BE); NORTHEASTERN UNIVERSITY, Boston, MA (US)

(21) Appl. No.: 15/552,177

(22) PCT Filed: Feb. 23, 2016
(86) PCT No.: PCT/EP2016/053792

§ 371 Date: Aug. 18, 2017

(30) Foreign Application Priority Data
Feb. 23, 2015 (EP) 15156159.4
Sep. 3, 2015 (EP) 15183766.3

(51) Int. Cl. A61K 9/107; A61K 47/06; A61K 39/39; A61K 9/00

(52) U.S. Cl. CPC A61K 9/1075; A61K 9/0019; A61K 47/06; A61K 39/39; A61K 2039/55566

(57) ABSTRACT

Oil-in-water emulsions with small droplet sizes can be formed without requiring either microfluidisation or heating to cause phase inversion, but rather by simple mixing of a pre-mixed composition of oil and surfactant with aqueous material. The oil/surfactant compositions can be mixed with an excess volume of aqueous material to spontaneously form an oil-in-water emulsion with droplets having a diameter<250 nm which shows good adjuvant activity. In general the oil makes up more than 50% by volume of the oil/surfactant composition, and the surfactant makes up the remainder.

### TECHNICAL FIELD

This invention relates to improved methods of manufacturing oil-in-water emulsions having small oil droplet particle sizes e.g. which are useful as vaccine adjuvants.

### BACKGROUND ART

The vaccine adjuvant known as ‘MF59’ [1-3] is a submicron oil-in-water emulsion of squalene, polysorbate 80 (also known as Tween 80), and sorbitan trioleate (also known as Span 85). It may also include citrate ions e.g. 10 mM sodium citrate buffer. The composition of the emulsion by volume can be about 5% squalene, about 0.5% Tween 80 and about 0.5% Span 85. The adjuvant and its production are described in more detail in references 4 (chapter 10), 5 (chapter 12) and 6 (chapter 19). As described in reference 7, it is manufactured on a commercial scale by dispersing Span 85 in the squalene, dispersing Tween 80 in an aqueous phase (citrate buffer), then mixing these two phases to form a coarse emulsion which is then microfluidised. The emulsion is prepared at double-strength and is diluted 1:1 (by volume) with the relevant vaccine.

The emulsion adjuvant known as ‘ASO3’ [8] is prepared by mixing an oil mixture (consisting of squalene and α-tocopherol) with an aqueous phase (Tween 80 and buffer), followed by microfluidisation [9]. It is also prepared at double-strength.

The emulsion adjuvant known as ‘AF03’ is prepared by cooling a pre-heated water-in-oil emulsion until it crosses its emulsion phase inversion temperature, at which point it thermoreversibly converts into an oil-in-water emulsion [10]. The ‘AF03’ emulsion includes squalene, sorbitan oleate, polyoxyethylene cetostearyl ether and mannitol. The mannitol, cetostearyl ether and a phosphate buffer are mixed in one container to form an aqueous phase, while the sorbitan ester and squalene are mixed in another container to form an oily component. The aqueous phase is added to the oily component and the mixture is then heated to ˜60° C. and cooled to provide the final emulsion. The emulsion is initially prepared with a composition of 32.5% squalene, 4.8% sorbitan oleate, 6.2% polyoxyethylene cetostearyl ether and 6% mannitol, which is at least 4× final strength.

As demonstrated above, previous methods known in the art for producing emulsions suitable for use as adjuvants require either vigorous mechanical processes (such as homogenisation and microfluidization) or relatively high temperatures (for example in a phase inversion temperature process) in order achieve the small oil droplet sizes required for adjuvant activity. The use of these processes is associated with several disadvantages e.g. high manufacturing costs.

### SUMMARY OF THE INVENTION

Accordingly, it is an object of the present invention to provide further and improved (e.g. simpler) methods for the production of submicron oil-in-water emulsions. In particular, it is an object of the present invention to provide methods that are suitable for use on a commercial scale and which do not require the use of processes involving vigorous mechanical treatment or significantly elevated temperatures.

The inventors have discovered that oil-in-water emulsions with small droplet sizes can be formed without requiring either microfluidisation or heating to cause phase inversion, but rather by simple mixing of a pre-mixed composition of oil and surfactant with aqueous material. The oil/surfactant compositions of the invention can be mixed with an excess volume of aqueous material to spontaneously form an oil-in-water emulsion with submicron oil droplets (and even with droplets having a diameter<220 nm, suitable for filter sterilisation) which shows good adjuvant activity.

In a first aspect, the invention provides an oil/surfactant composition suitable for preparing an oil-in-water emulsion adjuvant having an average oil particle diameter of less than 220 nm, said composition consisting essentially of an oil component and a surfactant component, wherein the oil component makes up 51-85% by volume of the composition. The phrase “suitable for preparing an oil-in-water emulsion adjuvant” means that the oil/surfactant composition can, when mixed (e.g. when mixed manually by a human) with an excess volume of surfactant-free aqueous material (e.g. with a 19× volume excess of 10 mM citrate buffer, pH 6.5, thus providing a 20-fold dilution), form an oil-in-water emulsion having the specified characteristics.

In a second aspect, the invention provides an oil/surfactant composition suitable for preparing an oil-in-water emulsion adjuvant having an average oil particle diameter of less than 220 nm, said composition consisting essentially of an oil component and a surfactant component, wherein the oil component makes up more than 50% by volume of the composition, and wherein the surfactant component consists of substantially equal volumes of two surfactants.

In a third aspect, the invention provides an oil/surfactant composition suitable for preparing an oil-in-water emulsion adjuvant having an average oil particle diameter of less than 220 nm, said composition consisting essentially of an oil component and a surfactant component, wherein the oil component makes up more than 50% by volume of the composition, and wherein the surfactant component has a HLB between 8 and 10.

In a fourth aspect, the invention provides an oil/surfactant composition suitable for preparing an oil-in-water emulsion adjuvant having an average oil particle diameter within the range of 140-200 nm, said composition consisting essentially of an oil component and a surfactant component, wherein the oil component makes up more than 50% by volume of the composition.

In a fifth aspect, the invention provides an oil/surfactant composition suitable for preparing an oil-in-water emulsion adjuvant having an average oil particle diameter within the range of 140-175 nm, said composition consisting essentially of an oil component and a surfactant component, wherein the oil component makes up more than 50% by volume of the composition.

In a sixth aspect the invention provides a method of forming an oil-in-water emulsion having an average oil particle diameter of less than 220 nm and comprising an oil component, an aqueous component, and a surfactant component, said method comprising: (i) providing an oil/surfactant composition according to any of the first five aspects of the invention; (ii) providing an aqueous component; (iii) combining the oil/surfactant composition with a volume excess of the aqueous component, to form a diluted composition; and (iv) gently mixing the diluted composition to form the oil-in-water emulsion.

The invention also provides oil-in-water emulsions obtainable by this method, along with their use in medicine e.g. for use as an immunological adjuvant. The invention also provides lyophilisates of oil-in-water emulsions obtainable by this method.

In a further aspect the invention provides an immunogenic composition comprising (i) an oil-in-water emulsion of the invention, and (ii) an immunogen component. Similarly, the invention provides a process for preparing an immunogenic composition, the process comprising mixing an oil-in-water emulsion according to the present invention with an immunogen component.

In another aspect the invention provides a kit comprising: (i) an oil/surfactant composition according to the invention; (ii) an aqueous component; and optionally (iii) instructions for combining the oil/surfactant composition and aqueous component.

In some embodiments, the oil/surfactant composition and/or the aqueous component may comprise an immunogen component.

In a further aspect the invention provides a process for preparing a kit comprising the steps of: (i) providing an oil/surfactant composition according to the present invention; and (ii) packaging the composition into a kit as a kit component together with an aqueous component; and optionally (iii) packaging an immunogen component into the kit as a kit component together with the oil/surfactant composition and the aqueous component.

The invention also provides a kit comprising: an oil-in-water emulsion according to the present invention; and an immunogen component. Similarly, the invention provides a process for preparing a kit comprising the steps of: (i) providing an oil-in-water emulsion according to the present invention; and (ii) packaging the emulsion into a kit as a kit component together with a separate immunogen component.

The present invention also provides a dry material (e.g. a lyophilisate) which, when reconstituted with an aqueous component, provides an oil-in-water emulsion according to the invention.

The invention also provides a method for preparing a dried emulsion, comprising: (i) obtaining an oil-in-water emulsion of the invention; and (ii) drying the oil-in-water emulsion to provide the dried emulsion. This dried material can be reconstituted into an emulsion of the invention by combining it with a suitable aqueous component. Suitable drying techniques are discussed below.

The present invention also provides a kit for preparing an oil-in-water emulsion according to the present invention, wherein the kit comprises: (i) a dried emulsion according to the invention; and (ii) an aqueous component, for mixing with the dried emulsion in order to provide an oil-in-water emulsion.

### Oil/Surfactant Compositions

According to the invention, processes for preparing oil-in-water emulsions make use of an oil/surfactant composition. This composition is a mixture of an oil component and a surfactant component, examples of which are discussed in more detail below. The oil(s) and surfactant(s) in these components are ideally miscible in each other in the composition. The composition may be an oil/surfactant dispersion, and if the oil and surfactant phases are fully miscible in each other the composition will be in the form of an oil/surfactant solution.

Because emulsions of the invention are intended for pharmaceutical use, the oil(s) and the surfactant(s) in the composition will typically be metabolisable (biodegradable) and biocompatible. If only one of the two components is metabolisable and biocompatible, it should be the oil component.

The composition ideally consists essentially of an oil component and a surfactant component. In some embodiments, however, the composition can include component(s) in addition to the oil and surfactant components. When further components are included, they should form less than 15% of the composition (by weight), more preferably less than 10%. For instance, in some embodiments the composition can include one or more pharmacologically active agent(s), which will usually be lipophilic. Typical lipophilic agents have a positive log P value (partition coefficient measured in 1-octanol and water) at pH 7.4 and 37° C. e.g. they may have a log P value≧1, ≧2, ≧3, ≧4, ≧5, ≧6, etc.

Oil/surfactant compositions of the invention should be substantially free of aqueous components, and they may be anhydrous.

The proportions of the oil component and the surfactant component can vary, provided that the composition will form an oil-in-water emulsion with submicron oil droplets when it is mixed with an excess volume of water (or other aqueous material). In general, however, the oil component makes up more than 50% by volume of the composition, and the surfactant component makes up the remainder. Usually the oil component will make up no more than 90% by volume of the composition, and more usually no more than 85% e.g. no more than 80% or no more than 75%. In the first aspect of the invention the oil component makes up 51-85% by volume of the composition, and the surfactant component will thus make up the remaining 15-49% by volume. The amount of oil may usefully be between 60-80% by volume, or between 65-75%, or between 68-72%. Useful oil proportions in the composition include, but are not limited to, 55%, 60%, 65%, 66⅔%, 70%, 75%, or 80% by volume. As shown below, composition with 70% oil forms a particularly good adjuvant emulsion.

A preferred oil/surfactant composition comprises squalene, sorbitan trioleate and polysorbate 80. More preferably it consists essentially of squalene, sorbitan trioleate and polysorbate 80 (and ideally the sorbitan trioleate and polysorbate 80 are present at equal volumes). According to certain embodiments the oil/surfactant composition can be one of the following (% by volume):

| | | | | | |
|---|---|---|---|---|---|
| Squalene | 70 | 75 | 80 | 70 | 70 |
| Sorbitan trioleate | 15 | 12.5 | 10 | 10 | 20 |
| Polysorbate 80 | 15 | 12.5 | 10 | 20 | 10 |

### The Oil Component

The composition includes an oil component which is formed from one or more oil(s). Suitable oil(s) include those from, for example, an animal (such as fish) or a vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolisable and so may be used, but this source is preferably avoided. The procedures for separation, purification, saponification and other means necessary for obtaining pure oils from animal sources are well known in the art.

The oil(s) in the composition's oil component will typically be biocompatible and biodegradable. Thus the oil component will not, under normal usage, harm a mammalian recipient when administered, and can be metabolised so that it does not persist.

Most fish contain metabolisable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. A preferred oil for use with the invention is squalene, which is a branched, unsaturated terpenoid ([(CH₃)₂C[═CHCH₂CH₂C(CH₃)]₂═CHCH₂-]₂; C₃₀H₅₀; 2,6,10,15,19,23-hexamethyl-2,6,10,14,18,22-tetracosahexaene; CAS RN 7683-64-9). Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ξ tocopherols can be used, but α-tocopherols are preferred. D-α-tocopherol and DL-α-tocopherol can both be used. A preferred α-tocopherol is DL-α-tocopherol. An oil combination comprising squalene and a tocopherol (e.g. DL-α-tocopherol) can be used.

As mentioned above, the oil component in a composition of the invention may include a combination of oils e.g. squalene and at least one further oil. Where the composition includes more than one oil, these can be present at various ratios e.g. between 1:5 and 5:1 by volume e.g. between 1:2 and 2:1, such as at equal volumes. Often, however, the oil component consists of a single oil, and the preferred oil is squalene.

### The Surfactant Component(s)

The composition includes a surfactant component which is formed from one or more surfactants(s). Usually it will consist of more than one surfactant, such as a mixture of two surfactants. In the invention's second aspect the surfactant component consists of substantially equal volumes of two surfactants.

The surfactant component can include various surfactants, including ionic, non-ionic and/or zwitterionic surfactants. The use of only non-ionic surfactants is preferred. The invention can thus use surfactants including, but not limited to: the polyoxyethylene sorbitan esters surfactants (commonly referred to as the Tweens or polysorbates), especially polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as polyoxyl 4 lauryl ether (Brij 30); polyoxyethylene-9-lauryl ether; sorbitan esters (commonly known as the Spans), such as sorbitan trioleate (Span 85) and sorbitan monolaurate; polyoxyethylene lauryl ether (Emulgen 104P). Many examples of pharmaceutically acceptable surfactants are known in the art for use in the composition and thus in the final emulsion e.g. see ‘Handbook of Pharmaceutical Excipients’ (eds. Rowe, Sheskey, & Quinn; 6th edition, 2009).

The surfactant(s) in the composition's surfactant component are preferably biocompatible and biodegradable. Thus the surfactant component will not, under normal usage, harm a mammalian recipient when administered, and can be metabolised so that it does not persist.

Two preferred surfactants for forming the surfactant component, either individually or in combination with at least one other surfactant (such as in combination with each other) are polysorbate 80 (‘Tween™ 80’) and sorbitan triolcate (‘Span™ 85’).

Surfactants can be classified by their ‘HLB’ (Griffin's hydrophile/lipophile balance), where a HLB in the range 1-10 generally means that the surfactant is more soluble in oil than in water, whereas a HLB in the range 10-20 means that the surfactant is more soluble in water than in oil. HLB values are readily available for surfactants of interest e.g. polysorbate 80 has a HLB of 15.0 and sorbitan trioleate has a HLB of 1.8.

When two or more surfactants are blended, the resulting HLB of the blend is easily calculated by the weighted average e.g. a 70/30 wt % mixture of polysorbate 80 and sorbitan triolcate has a HLB of (15.0×0.70)+(1.8×0.30) i.e. 11.04.

In general, and in particular for the invention's third aspect, the surfactant component has a HLB between 8 and 10. This can be achieved using a single surfactant (e.g. Brij 30, having a HLB of 9.7; Emulgen 104P, 9.6; Ethylan 254, 9.8; Plurafac RA30, 9.0; oleth 5 polyethylene glycol ether of oleyl alcohol, 8.8; Hetoxide C-16, 8.6; polysorbate 61, 9.6; polyoxyl stearate, 9.7; sorbitan monolaurate, 8.6) or, more typically, using a mixture of surfactants (e.g. of two surfactants, such as polysorbate 80 and sorbitan triolcate).

Where the surfactant component includes more than one surfactant then at least one of them will typically have a HLB of at least 10 (e.g. in the range 12-16, or 13 to 17) and at least one has a HLB below 10 (e.g. in the range of 1-9, or 1-4). For instance, the surfactant component of the composition can include polysorbate 80 and sorbitan triolcate. In some embodiments the surfactant component comprises a first surfactant having an HLB value of from 1 to 5 and a second surfactant having an HLB value of from 13 to 17.

One preferred surfactant component consists of a mixture of polysorbate 80 and sorbitan trioleate. By varying the volume ratio of these two surfactants a HLB of 8 can be achieved with a 44:56 volume mixture (excess sorbitan trioleate), and a HLB of 10 can be achieved with a 59:41 mixture (excess polysorbate 80). Preferably the two surfactants are used at equal volumes (i.e. in accordance with the second aspect of the invention) which, in weight terms, gives a mixture with 53.2% polysorbate 80 and 46.8% sorbitan trioleate, and thus a HLB of 8.8.

According to certain embodiments the useful surfactant proportions in the composition include, but are not limited to (% by volume of the oil/surfactant composition): no more than 49%, 45%, 40%, 35%, 30%, 29%, 28%, 27%, 26%, 25%, 24%, 23%, 22%, 21%, 20%, 19%, 18%, 17%, 16% or 15%.

Another useful surfactant component, as seen in ‘AF03’, may be made from sorbitan monooleate (which has HLB 4.3) and polyoxyethylene cetostearyl ether (HLB 13.5).

Where an oil/surfactant composition includes a surfactant having a HLB above 8 then the concentration of that surfactant is preferably at least 400× higher than its critical micelle concentration (CMC) e.g. at least 500× higher, 600× higher, 800× higher, etc. based on the final emulsion. If the oil/surfactant composition is diluted 20-fold with an aqueous component, for instance, the concentration of the surfactant in the oil-surfactant composition would be at least 8000× higher than its CMC, which would be diluted to 400×.

### Optional Disclaimer

In some embodiments the invention does not encompass: (i) an oil/surfactant composition consisting of 70% by volume squalene, 20% by volume sorbitan trioleate, and 10% by volume polysorbate 80; or (ii) an oil/surfactant composition consisting of 60% by volume squalene, 20% by volume sorbitan trioleate, and 20% by volume polysorbate 80.

### The Aqueous Component

According to the invention, processes for preparing emulsions make use of an aqueous component, which is mixed with an oil/surfactant composition of the invention. This aqueous component can be plain water (e.g. w.f.i.) or can include further components e.g. solutes. For instance, it preferably includes salts, which can be used to influence tonicity and/or to control pH. For instance, the salts can form a pH buffer e.g. citrate or phosphate salts, such as sodium salts. Typical buffers include: a phosphate buffer; a Tris buffer; a borate buffer; a succinate buffer; a histidine buffer; or a citrate buffer. Where a buffered aqueous component is used the buffer will typically be included in the 1-20 mM range.

The pH of the aqueous component will preferably be buffered between 6.0-8.0, preferably about 6.2 to about 6.8. In an exemplary embodiment, the buffer is 10 mM citrate buffer with a pH at 6.5. The aqueous component may comprise pickering agents such as mannitol to reduce superficial tension.

The aqueous component can include solutes for influencing tonicity and/or osmolality. The tonicity can be selected to be isotonic with human tissues. To control tonicity, the emulsion may comprise a physiological salt, such as a sodium salt. Sodium chloride (NaCl), for example, may be used at about 0.9% (w/v) (physiological saline). Other salts that may be present include potassium chloride, potassium dihydrogen phosphate, disodium phosphate, magnesium chloride, calcium chloride, etc. Non-ionic tonicifying agents can also be used to control tonicity. Monosaccharides classified as aldoses such as glucose, mannose, arabinose, and ribose, as well as those classified as ketoses such as fructose, sorbose, and xylulose can be used as non-ionic tonicifying agents in the present invention. Disaccharides such a sucrose, maltose, trehalose, and lactose can also be used. In addition, alditols (acyclic polyhydroxy alcohols, also referred to as sugar alcohols) such as glycerol, mannitol, xylitol, and sorbitol are non-ionic tonicifying agents useful in the present invention. Non-ionic tonicity modifying agents can be present at a concentration of from about 0.1% to about 10% or about 1% to about 10%, of the aqueous component depending upon the agent that is used.

The aqueous component ideally has a pH between 5.5 and 8.5 e.g. between 6.0 and 8.0, or between 6.5 and 7.5. This pH range maintains compatibility with normal physiological conditions and, in certain instances, may be required in order to ensure stability of certain components of the emulsion.

Preferably, the aqueous component is substantially free from oil(s). Thus, on mixing with the oil/surfactant composition to form an emulsion, substantially all of the oil in the emulsion should be sourced from the oil/surfactant composition. Preferably, the aqueous component is also substantially free from surfactant(s). Thus, on mixing with the oil/surfactant composition to form an emulsion, substantially all of the surfactant in the emulsion should be sourced from the oil/surfactant composition. Most preferably, the aqueous component is substantially free from both oil(s) and surfactant(s).

In some embodiments the aqueous phase may comprise an immunogen component.

### Mixing

Unlike MF59 and AS03, emulsions of the invention can be prepared without requiring the use of homogenisers or microfluidisers. Unlike AF03, emulsions of the invention can be prepared without requiring heating up to >50° C. Instead, mixing the oil/surfactant composition with the aqueous phase can lead to spontaneous formation of a submicron emulsion even with only gentle agitation/mixing (e.g. by hand, such as by simple manual inversion).

Thus the sixth aspect of the invention provides a method for forming an oil-in-water emulsion comprising: (i) providing an oil/surfactant composition according to the invention; (ii) providing an aqueous component; (iii) combining the oil/surfactant composition with a volume excess of the aqueous component, to form a diluted composition; and (iv) gently mixing the diluted composition. Steps (iii) and (iv) may take place simultaneously.

Step (iii) can take place by simple mixing of the oil/surfactant composition with the aqueous component. Preferably it is achieved by adding the oil/surfactant composition into the aqueous component. Step (iii) may sometimes comprise two separate steps: (a) mixing equal volumes of oil/surfactant composition and aqueous component; and (b) diluting the mixture of oil/surfactant composition and aqueous component with a further volume of an aqueous component to form the diluted composition. The steps (a) and (b) are preferably each achieved by adding the oil/surfactant-containing material into an aqueous component.

The mixing in step (iv) can be carried out without requiring any shear pressure, without using rotor/stator mixing, at normal pressures, and without circulating components through a pump. It can be performed in the absence of mechanical agitation. It can be performed in the absence of thermal inversion.

The mixture of the composition and the aqueous component can be gently agitated/mixed in order to form an oil-in-water emulsion. The gentle mixing is provided by means other than homogenization, microfiltration, microfluidisation, sonication (or other high shear or high energy processes) or a phase inversion temperature process in which the temperature of the emulsion is raised until it inverts. Suitably, the gentle agitation may comprise inversion of the mixture by hand, or it may comprise stirring, or it may comprise mixing by passing through a syringe, or it may comprise any similar process. Overall, mixing is achieved by applying controlled minimal dispersion force. Inclusion of mechanical mixing components (e.g. magnetic stirring bars) is ideally avoided.

The step of combining the oil/surfactant composition and aqueous component can take place below 55° C. e.g. anywhere in the range of 5-50°, for example between 10-20° C., between 20-30° C., between 30-50° C., or between 40-50° C. The process can usefully take place at room temperature i.e. about 20-25° C. This step is ideally performed at below 30° C. e.g. in the range of 15-29° C. The composition and/or the aqueous phase are preferably equilibrated to the desired temperature before being mixed. For instance, the two components could be equilibrated to 40° C. and then be mixed. After mixing, the mixture can be maintained at a temperature below 55° C. while the emulsion forms. Preferably, the oil/surfactant composition and/or aqueous component are heated before mixing and held at the desired temperature (below 55° C.) until the mixing of the two components is complete and thereafter the temperature is reduced.

The oil/surfactant composition is mixed with a volume excess of the aqueous component, to ensure that an oil-in-water emulsion is formed (rather than a water-in-oil emulsion). As mentioned above, the aqueous component is preferably substantially free from surfactant(s) and/or oil(s). The process preferably uses the aqueous component at a volume excess of at least 4-fold to the oil/surfactant composition e.g. between 4-fold to 50-fold greater volume. Preferably the aqueous component has a volume which is 9× to 50× larger than the volume of the oil/surfactant composition. More preferably the excess is from 19× to 39× (by volume), thus giving a 20-fold to 40-fold dilution. A 19× excess can be particularly useful.

The method can be used at a lab or benchtop scale or at industrial scale. Thus the composition and/or aqueous phase may have a volume in the range of 1-100 mL, in the range of 100-1000 mL, in the range of 1-10 L, or even in the range of 10-100 L.

The method may further comprise the step of subjecting the oil-in-water emulsion to filter sterilisation. The filter sterilisation can take place at any suitable stage e.g. when placing the emulsion into containers (the fill stage), or prior to drying (which can be performed aseptically, to maintain a sterile emulsion during and after drying).

### Oil-in-Water Emulsions

The invention provides oil-in-water emulsions obtainable by the method disclosed above. The oil particles in these emulsions have an average diameter of less than 220 nm, and in some embodiments within the range of 90-220 nm or 100-220 nm or 110-220 nm or 120-220 nm or 130-220 nm or 90-200 nm or 100-200 nm or 110-200 nm or 120-200 nm or 130-200 nm or 140-200 nm or even 140-175 nm, making them useful as immunological adjuvants. In general, diameters above 85 nm, but less than 220 nm, are preferred. Diameters of 140-200 nm are most preferred.

The average diameter of oil particles in an emulsion can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See also reference 11. Dynamic light scattering (DLS) is the preferred method by which oil particle diameters are determined. The preferred method for defining the average oil particle diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to an average diameter should be taken as an intensity-weighted average, and ideally the Z-average.

Droplets within emulsions of the invention preferably have a polydispersity index of less than 0.4. Polydispersity is a measure of the width of the size distribution of particles, and is conventionally expressed as the polydispersity index (PdI). A polydispersity index of greater than 0.7 indicates that the sample has a very broad size distribution and a reported value of 0 means that size variation is absent, although values smaller than 0.05 are rarely seen. It is preferred for oil droplets within an emulsion of the invention to be of a relatively uniform size. Thus oil droplets in emulsions preferably have a PdI of less than 0.35 e.g. less than 0.3, 0.275, 0.25, 0.225, 0.2, 0.175, 0.15, 0.125, or even less than 0.1. PdI values are easily provided by the same instrumentation which measures average diameter.

### Optional Disclaimer

In some embodiments the invention does not encompass an oil-in-water emulsion comprising squalene, sorbitan trioleate and polysorbate in a volume ratio 8.6:1:1 (i.e. as seen in the MF59 emulsion). In some instances this disclaimer applies only if the PdI of the emulsion is greater than 0.12.

### Downstream Processing

Oil-in-water emulsions of the invention can be filtered. This filtration removes any large oil droplets from the emulsion. Although small in number terms, these oil droplets can be large in volume terms and they can act as nucleation sites for aggregation, leading to emulsion degradation during storage. Moreover, this filtration step can achieve filter sterilization.

The particular filtration membrane suitable for filter sterilization depends on the fluid characteristics of the oil-in-water emulsion and the degree of filtration required. A filter's characteristics can affect its suitability for filtration of the emulsion. For example, its pore size and surface characteristics can be important, particularly when filtering a squalene-based emulsion. Details of suitable filtration techniques are available e.g. in reference 12.

The pore size of membranes used with the invention should permit passage of the desired droplets while retaining the unwanted droplets. For example, it should retain droplets that have a size of ≧1 μm while permitting passage of droplets<200 nm. A 0.2 μm or 0.22 μm filter is ideal, and can also achieve filter sterilization.

The emulsion may be prefiltered e.g. through a 0.45 μm filter. The prefiltration and filtration can be achieved in one step by the use of known double-layer filters that include a first membrane layer with larger pores and a second membrane layer with smaller pores. Double-layer filters are particularly useful with the invention. The first layer ideally has a pore size>0.3 μm, such as between 0.3-2 μm or between 0.3-1 μm, or between 0.4-0.8 μm, or between 0.5-0.7 μm. A pore size of ≦0.75 μm in the first layer is preferred. Thus the first layer may have a pore size of 0.6 μm or 0.45 μm, for example. The second layer ideally has a pore size which is less than 75% of (and ideally less than half of) the first layer's pore size, such as between 25-70% or between 25-49% of the first layer's pore size e.g. between 30-45%, such as ⅓ or 4/9, of the first layer's pore size. Thus the second layer may have a pore size<0.3 μm, such as between 0.15-0.28 μm or between 0.18-0.24 μm e.g. a 0.2 μm or 0.22 μm pore size second layer. In one example, the first membrane layer with larger pores provides a 0.45 μm filter, while the second membrane layer with smaller pores provides a 0.22 μm filter.

The filtration membrane and/or the prefiltration membrane may be asymmetric. An asymmetric membrane is one in which the pore size varies from one side of the membrane to the other e.g. in which the pore size is larger at the entrance face than at the exit face. One side of the asymmetric membrane may be referred to as the “coarse pored surface”, while the other side of the asymmetric membrane may be referred to as the “fine pored surface”. In a double-layer filter, one or (ideally) both layers may be asymmetric.

The filtration membrane may be porous or homogeneous. A homogeneous membrane is usually a dense film ranging from 10 to 200 μm. A porous membrane has a porous structure. In one embodiment, the filtration membrane is porous. In a double-layer filter, both layers may be porous, both layers may be homogenous, or there may be one porous and one homogenous layer. A preferred double-layer filter is one in which both layers are porous.

In one embodiment, the oil-in-water emulsions of the invention are prefiltered through an asymmetric, hydrophilic porous membrane and then filtered through another asymmetric hydrophilic porous membrane having smaller pores than the prefiltration membrane. This can use a double-layer filter.

The filter membrane(s) may be autoclaved prior to use to ensure that it is sterile.

Filtration membranes are typically made of polymeric support materials such as PTFE (poly-tetra-fluoro-ethylene), PES (polyethersulfone), PVP (polyvinyl pyrrolidone). PVDF (polyvinylidene fluoride), nylons (polyamides), PP (polypropylene), celluloses (including cellulose esters), PEEK (polyetheretherketone), nitrocellulose, etc. These have varying characteristics, with some supports being intrinsically hydrophobic (e.g. PTFE) and others being intrinsically hydrophilic (e.g. cellulose acetates). However, these intrinsic characteristics can be modified by treating the membrane surface. For instance, it is known to prepare hydrophilized or hydrophobized membranes by treating them with other materials (such as other polymers, graphite, silicone, etc.) to coat the membrane surface e.g. see section 2.1 of reference 13. In a double-layer filter the two membranes can be made of different materials or (ideally) of the same material.

During filtration, the emulsion may be maintained at a temperature of 40° C. or less, e.g. 30° C. or less, to facilitate successful sterile filtration. Some emulsions may not pass through a sterile filter when they are at a temperature of greater than 40° C.

It is advantageous to carry out the filtration step within 24 hours, e.g. within 18 hours, within 12 hours, within 6 hours, within 2 hours, within 30 minutes, of producing the emulsion because after this time it may not be possible to pass the second emulsion through the sterile filter without clogging the filter, as discussed in reference 14.

Methods of the invention may be used at large scale. Thus a method may involve filtering a volume greater than 1 liter e.g. ≧5 liters, ≧10 liters, ≧20 liters, ≧50 liters, ≧100 liters, ≧250 liters, etc.

In some embodiments an emulsion which has been prepared according to the invention can be subjected to microfluidisation. Thus, for instance, the invention can be used prior to microfluidisation to reduce the degree of microfluidising which is required for giving a desired result. Thus, if desired, microfluidisation can be used but the overall shear forces imparted on the emulsion can be reduced.

Oil-in-water emulsions of the invention can be dried (optionally after being filtered, as discussed above). Drying can conveniently be achieved by lyophilisation, but other techniques can also be used e.g. spray drying. These dried emulsions can be mixed with an aqueous component to provide once again an emulsion of the invention. Thus the invention provides a dry material (e.g. a lyophilisate) which, when reconstituted with an aqueous component, provides an oil-in-water emulsion of the invention.

As used herein, “dry material” and “dried material” refer to material which is substantially free of water or substantially free of an aqueous phase (e.g. it is substantially anhydrous). The dry material will usually take the form of a powder or a cake.

The invention also provides processes for preparing said dry material by preparing an oil-in-water emulsion according to the invention and subjecting it to a drying process. Suitably the emulsion is combined with (or already includes) one or more lyophilisation stabilizers prior to lyophilisation. The emulsion may also be combined with at least one immunogen component prior to drying, optionally in addition to one or more lyophilisation stabilizers.

A dry emulsion can be provided with other components in liquid form (e.g. an immunogen and/or an aqueous component). These components can be mixed in order to reactivate the dry component and give a liquid composition for administration to a patient. A dried component will typically be located within a vial rather than a syringe.

A lyophilised component (e.g. the emulsion) may include lyophilisation stabilizers. These stabilizers include substances such as sugar alcohols (e.g. mannitol, etc.) or simple saccharides such as disaccharides and trisaccharides. Lyophilisation stabilizers are preferably small saccharides such as disaccharides. They preferably include saccharide monomers selected from glucose, fructose and galactose, and glucose-containing disaccharides and fructose-containing disaccharides are particularly preferred. Examples of preferred disaccharides include sucrose (containing glucose and fructose), trehalose (containing two glucose monosaccharides) and maltulose (containing glucose and fructose), more preferably sucrose. such as lactose, sucrose or mannitol, as well as mixtures thereof e.g. lactose/sucrose mixtures, sucrose/mannitol mixtures, etc.

An advantage of the oil-in-water emulsions of the invention and the methods for making them according to the invention is that when they are reactivated with an aqueous component following drying, the resultant oil-in-water emulsion can retain its original properties from prior to drying (e.g. its average oil particle diameter).

### Immunogens

Although it is possible to administer oil-in-water emulsion adjuvants on their own to patients (e.g. to provide an adjuvant effect for an immunogen that has been separately administered to the patient), it is more usual to admix the adjuvant with an immunogen prior to administration, to form an immunogenic composition e.g. a vaccine. Mixing of emulsion and immunogen may take place extemporaneously, at the time of use, or can take place during vaccine manufacture, prior to filling. The emulsions of the invention can be used in either situation.

Various immunogens can be used with oil-in-water emulsions, including but not limited to: viral antigens, such as viral surface proteins; bacterial antigens, such as protein and/or saccharide antigens; fungal antigens; parasite antigens; and tumor antigens. The invention is particularly useful for vaccines against influenza virus, HIV, hookworm, hepatitis B virus, herpes simplex virus (and other herpesviridae), rabies, respiratory syncytial virus, cytomegalovirus, Staphylococcus aureus. chlamydia, SARS coronavirus, varicella zoster virus, Streptococcus pneumoniae, Neisseria meningitidis, Mycobacterium tuberculosis, Bacillus anthracis, Epstein Barr virus, human papillomavirus, malaria, etc. For example:

Influenza Virus Antigens.

These may take the form of a live virus or an inactivated virus. Where an inactivated virus is used, the vaccine may comprise whole virion, split virion, or purified surface antigens (including hemagglutinin and, usually, also including neuraminidase). Influenza antigens can also be presented in the form of virosomes. The antigens may have any hemagglutinin subtype, selected from H1, H2, H3, H4, H5, H6, H7, H8, H9, H10, H11, H12, H13, H14, H15 and/or H16. Vaccine may include antigen(s) from one or more (e.g. 1, 2, 3, 4 or more) influenza virus strains, including influenza A virus and/or influenza B virus, e.g. a monovalent A/H5N1 or A/H1N1 vaccine, or a trivalent A/H1N1+A/H3N2+B vaccine. The vaccines can be for seasonal or pandemic use. The influenza virus may be a reassortant strain, and may have been obtained by reverse genetics techniques [e.g. 15-19]. Thus the virus may include one or more RNA segments from a A/PR8/34 virus (typically 6 segments from A/PR/8/34, with the HA and N segments being from a vaccine strain, i.e. a 6:2 reassortant). The viruses used as the source of the antigens can be grown either on eggs (e.g. embryonated hen eggs) or on cell culture. Where cell culture is used, the cell substrate will typically be a mammalian cell line, such as MDCK; CHO; 293T; BHK; Vero; MRC-5; PER.C6; WI-38; etc. Preferred mammalian cell lines for growing influenza viruses include: MDCK cells [20-23], derived from Madin Darby canine kidney; Vero cells [24-26], derived from African green monkey kidney; or PER.C6 cells [27], derived from human embryonic retinoblasts. Where virus has been grown on a mammalian cell line then the composition will advantageously be free from egg proteins (e.g. ovalbumin and ovomucoid) and from chicken DNA, thereby reducing allergenicity. Unit doses of vaccine are typically standardized by reference to hemagglutinin (HA) content, typically measured by SRID. Existing vaccines typically contain about 15 μg of HA per strain, although lower doses can be used, particularly when using an adjuvant. Fractional doses such as 6 (i.e. 7.5 μg HA per strain), ¼ and ⅛ have been used [28,29], as have higher doses (e.g. 3× or 9× doses [30,31]). Thus vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 15, about 10, about 7.5, about 5, about 3.8, about 3.75, about 1.9, about 1.5, etc. per strain.

Human immunodeficiency virus, including HIV-1 and HIV-2. The antigen will typically be an envelope antigen.

Hepatitis B Virus Surface Antigens.

This antigen is preferably obtained by recombinant DNA methods e.g. after expression in a Saccharomyces cerevisiae yeast. Unlike native viral HBsAg, the recombinant yeast-expressed antigen is non-glycosylated. It can be in the form of substantially-spherical particles (average diameter of about 20 nm), including a lipid matrix comprising phospholipids. Unlike native HBsAg particles, the yeast-expressed particles may include phosphatidylinositol. The HBsAg may be from any of subtypes ayw1, ayw2, ayw3, ayw4, ayr, adw2, adw4, adrq− and adrq+.

Hookworm, particularly as seen in canines (Ancylostoma caninum). This antigen may be recombinant Ac-MTP-1 (astacin-like metalloprotease) and/or an aspartic hemoglobinase (Ac-APR-1), which may be expressed in a baculovirus/insect cell system as a secreted protein [32,33].

Herpes simplex virus antigens (HSV). A preferred HSV antigen for use with the invention is membrane glycoprotein gD. It is preferred to use gD from a HSV-2 strain (‘gD2’ antigen). The composition can use a form of gD in which the C-terminal membrane anchor region has been deleted [34] e.g. a truncated gD comprising amino acids 1-306 of the natural protein with the addition of aparagine and glutamine at the C-terminus. This form of the protein includes the signal peptide which is cleaved to yield a mature 283 amino acid protein. Deletion of the anchor allows the protein to be prepared in soluble form. The invention can also be used with other herpesviridae, such as varicella-zoster virus (VZV), Epstein-Barr virus (EBV), or human cytomegalovirus (hCMV). An anti-hCMV composition can include a glycoprotein B (gB) antigen in some embodiments, or can include one or more of the gH, gL and gO antigens.

Human papillomavirus antigens (HPV). Preferred HPV antigens for use with the invention are L1 capsid proteins, which can assemble to form structures known as virus-like particles (VLPs). The VLPs can be produced by recombinant expression of L1 in yeast cells (e.g. in S. cerevisiae) or in insect cells (e.g. in Spodoptera cells, such as S. frugiperda, or in Drosophila cells). For yeast cells, plasmid vectors can carry the L1 gene(s); for insect cells, baculovirus vectors can carry the L1 gene(s). More preferably, the composition includes L1 VLPs from both HPV-16 and HPV-18 strains. This bivalent combination has been shown to be highly effective [35]. In addition to HPV-16 and HPV-18 strains, it is also possible to include L1 VLPs from HPV-6 and HPV-II strains. The use of oncogenic HPV strains is also possible. A vaccine may include between 20-60 g/ml (e.g. about 40 μg/ml) of L1 per HPV strain.

Anthrax Antigens.

Anthrax is caused by Bacillus anthracis. Suitable B. anthracis antigens include A-components (lethal factor (LF) and edema factor (EF)), both of which can share a common B-component known as protective antigen (PA). The antigens may optionally be detoxified. Further details can be found in references [36 to 38].

Malaria Antigens.

A composition for protecting against malaria can include a portion of the P. falciparum circumsporozoite protein from the organism's pre-erythrocytic stage. The C-terminal portion of this antigen can be expressed as a fusion protein with HBsAg, and this fusion protein can be co-expressed with HBsAg in yeast such that the two proteins assemble to form a particle.

Rabies.

Compositions for protecting against rabies will generally include an inactivated rabies virus virion, as seen in products such as RABIPUR, RABIVAC, and VERORAB.

S. aureus Antigens.

A variety of S. aureus antigens are known. Suitable antigens include capsular saccharides (e.g. from a type 5 and/or type 8 strain) and proteins (e.g. IsdB, Hla, etc.). Capsular saccharide antigens are ideally conjugated to a carrier protein.

S. pneumoniae Antigens.

A variety of S. pneumoniae antigens are known. Suitable antigens include capsular saccharides (e.g. from one or more of serotypes 1, 4, 5, 6B, 7F, 9V, 14, 18C, 19F, and/or 23F) and proteins (e.g. pneumolysin, detoxified pneumolysin, polyhistidine triad protein D (PhtD), etc.). Capsular saccharide antigens are ideally conjugated to a carrier protein.

Meningococcal Antigens.

Neisseria meningitidis is a cause of bacterial meningitis. Suitable meningococcal antigens include conjugated capsular saccharides (particularly for serogroups A, C, W135, X and/or Y), recombinant proteins (e.g. factor H binding protein) and/or outer membrane vesicles.

Cancer Antigens.

A variety of tumour-specific antigens are known. The invention may be used with antigens that elicit an immunotherapeutic response against lung cancer, melanoma, breast cancer, prostate cancer, etc.

A solution of the immunogen will normally be mixed with the emulsion e.g. at a 1:1 volume ratio. This mixing can either be performed by a vaccine manufacturer, prior to filling, or can be performed at the point of use, by a healthcare worker. As noted below, however, an alternative formulation includes both immunogen and emulsion in dried form in a single container for reconstitution.

### Uses of the Oil-in-Water Emulsions of the Invention

Oil-in-water emulsions of the invention are suitable for use as immunological adjuvants. Suitably these adjuvants are administered as part of a vaccine. Thus the invention provides an immunogenic composition, such as a vaccine, comprising (i) an oil-in-water emulsion of the invention, and (ii) an immunogen component. These can be made by mixing an oil-in-water emulsion of the invention with an immunogen component.

The invention also provides kits comprising: an oil-in-water emulsion of the invention; and an immunogen component. The invention also provides kits comprising: an oil/surfactant composition; an aqueous component; and an immunogen component. Mixing of the kit components provides an immunogenic composition of the invention.

The invention also provides kits comprising an oil/surfactant composition of the invention and an aqueous component, either or both of which includes an immunogen. Mixing of the kit components provides an immunogenic composition of the invention.

Although it is possible to administer oil-in-water emulsion adjuvants on their own to patients (e.g. to provide an adjuvant effect for an immunogen that has been separately administered), it is more usual to admix the adjuvant with an immunogen prior to administration, to form an immunogenic composition e.g. a vaccine. Mixing of emulsion and immunogen may take place extemporaneously, at the time of use, or can take place during vaccine manufacture, prior to filling.

Overall, therefore, the invention can be used when preparing mixed vaccines or when preparing kits for mixing as discussed above. Where mixing takes place during manufacture then the volumes of bulk immunogen and emulsion that are mixed will typically be greater than 1 liter e.g. ≧5 liters, ≧10 liters, ≧20 liters, ≧50 liters, ≧100 liters, ≧250 liters, etc. Where mixing takes place at the point of use then the volumes that are mixed will typically be smaller than 1 milliliter e.g. ≦0.6 ml, ≦0.5 ml, ≦0.4 ml, ≦0.3 ml, ≦0.2 ml, etc. In both cases it is usual for substantially equal volumes of emulsion and immunogen solution to be mixed i.e. substantially 1:1 (e.g. between 1.1:1 and 1:1.1, preferably between 1.05:1 and 1:1.05, and more preferably between 1.025:1 and 1:1.025). In some embodiments, however, an excess of emulsion or an excess of immunogen may be used [39]. Where an excess volume of one component is used, the excess will generally be at least 1.5:1 e.g. ≧2:1, ≧2.5:1, ≧3:1, ≧4:1, ≧5:1, etc.

Where an immunogen and an adjuvant are presented as separate components within a kit, they are physically separate from each other within the kit, and this separation can be achieved in various ways. For instance, the components may be in separate containers, such as vials. The contents of two vials can then be mixed when needed e.g. by removing the contents of one vial and adding them to the other vial, or by separately removing the contents of both vials and mixing them in a third container.

In another arrangement, one of the kit components is in a syringe and the other is in a container such as a vial. The syringe can be used (e.g. with a needle) to insert its contents into the vial for mixing, and the mixture can then be withdrawn into the syringe. The mixed contents of the syringe can then be administered to a patient, typically through a new sterile needle. Packing one component in a syringe eliminates the need for using a separate syringe for patient administration.

In another useful arrangement, the two kit components are held together but separately in the same syringe e.g. a dual-chamber syringe, such as those disclosed in references 40-47 etc. When the syringe is actuated (e.g. during administration to a patient) then the contents of the two chambers are mixed. This arrangement avoids the need for a separate mixing step at time of use.

The contents of the various kit components can all be in liquid form, but in some embodiments a dry emulsion might be included.

Vaccines are typically administered by injection, particularly intramuscular injection. Compositions of the invention are generally presented at the time of use as aqueous solutions or suspensions, and are ideally suitable for intramuscular injection. In some embodiments of the invention the compositions are in aqueous form from the packaging stage to the administration stage. In other embodiments, however, one or more components of the compositions may be packaged in dried (e.g. lyophilised) form, and an adjuvant for actual administration may be reconstituted when necessary. The emulsion may thus be distributed as a lyophilized cake, as discussed above.

One possible arrangement according to a preferred aspect of the present invention comprises a dried emulsion component in a vial and an immunogen component and/or aqueous component in a pre-filled syringe.

The present invention also provides an arrangement comprising a dried emulsion of the present invention and a separate liquid immunogen component.

Also provided by the present invention is a dried cake formed from the emulsion of the invention. The cake may be provided in combination with a separate aqueous phase. The arrangement may further comprise an immunogen component which may be in liquid or dried form.

The present invention also provides a dried mixture wherein the mixture comprises the emulsion of the present invention in combination with an immunogen component. Preferably the mixture is a lyophilized mixture. Reactivation of this mixture with an aqueous component provides an immunogenic composition of the invention.

The invention also provides a kit for preparing an oil-in-water emulsion of the invention, wherein the kit comprises an oil-in-water emulsion of the invention in dry form and an aqueous phase in liquid form. The kit may comprises two vials (one containing the dry emulsion and one containing the aqueous phase) or it may comprise one ready filled syringe and one vial e.g. with the contents of the syringe (the aqueous component) being used to reconstitute the contents of the vial (the dry emulsion) prior to administration to a subject. In embodiments of the invention the oil-in-water emulsion in dry form is combined with an immunogen component that is also in dry form.

If vaccines contain components in addition to emulsion and immunogen then these further components may be included in one of the two kit components according to embodiments of the invention, or may be part of a third kit component.

Suitable containers for mixed vaccines of the invention, or for individual kit components, include vials and disposable syringes. These containers should be sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. In one embodiment, a vial has a butyl rubber stopper. The vial may include a single dose of vaccine/component, or it may include more than one dose (a ‘multidose’ vial) e.g. 10 doses. In one embodiment, a vial includes 10×0.25 ml doses of emulsion. Preferred vials are made of colourless glass.

A vial can have a cap (e.g. a Luer lock) adapted such that a pre-filled syringe can be inserted into the cap, the contents of the syringe can be expelled into the vial (e.g. to reconstitute dried material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed.

Where a composition/component is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of adjuvant or vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield.

The emulsion may be diluted with a buffer prior to packaging into a vial or a syringe. Typical buffers include: a phosphate buffer, a Tris buffer; a borate buffer; a succinate buffer, a histidine buffer; or a citrate buffer. Dilution can reduce the concentration of the adjuvant's components while retaining their relative proportions e.g. to provide a “half-strength” adjuvant.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

Compositions made using the methods of the invention are pharmaceutically acceptable. They may include components in addition to the emulsion and the optional immunogen.

The composition may include a preservative such as thiomersal or 2-phenoxyethanol. It is preferred, however, that the adjuvant or vaccine should be substantially free from (i.e. less than 5 μg/ml) mercurial material e.g. thiomersal-free [48,49]. Vaccines and components containing no mercury are more preferred.

The pH of an aqueous immunogenic composition will generally be between 5.0 and 8.1, and more typically between 6.0 and 8.0 e.g. between 6.5 and 7.5. A process of the invention may therefore include a step of adjusting the pH of the adjuvant or vaccine prior to packaging or drying.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunization, or may include material for multiple immunizations (i.e. a ‘multidose’ kit). The inclusion of a preservative is preferred in multidose arrangements.

The compositions can be administered in various ways. The most preferred immunization route is by intramuscular injection (e.g. into the arm or leg), but other available routes include subcutaneous injection, intranasal [50-52], oral [53], intradermal [54,55], transcutaneous, transdermal [56], etc. Compositions suitable for intramuscular injection are most preferred.

Adjuvants or vaccines prepared according to the invention may be used to treat both children and adults. The patient may be less than 1 year old, 1-5 years old, 5-15 years old, 15-55 years old, or at least 55 years old. The patient may be elderly (e.g. ≧50 years old, preferably ≧65 years), the young (e.g. ≦5 years old), hospitalized patients, healthcare workers, armed service and military personnel, pregnant women, the chronically ill, immunodeficient patients, and people travelling abroad. The vaccines are not suitable solely for these groups, however, and may be used more generally in a population.

Adjuvants or vaccines of the invention may be administered to patients at substantially the same time as (e.g. during the same medical consultation or visit to a healthcare professional) other vaccines.

### General

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encephalopathics (TSEs), and in particular free from bovine spongiform encephalopathy (BSE). Overall, it is preferred to culture cells in the total absence of animal-derived materials.

### MODES FOR CARRYING OUT THE INVENTION

The examples set out below are for illustrative purposes and are not intended to limit the scope of the invention. The examples refer to the following Figures:

FIG. 1—ELISA results for 0.1 μg TIV adjuvanted study.

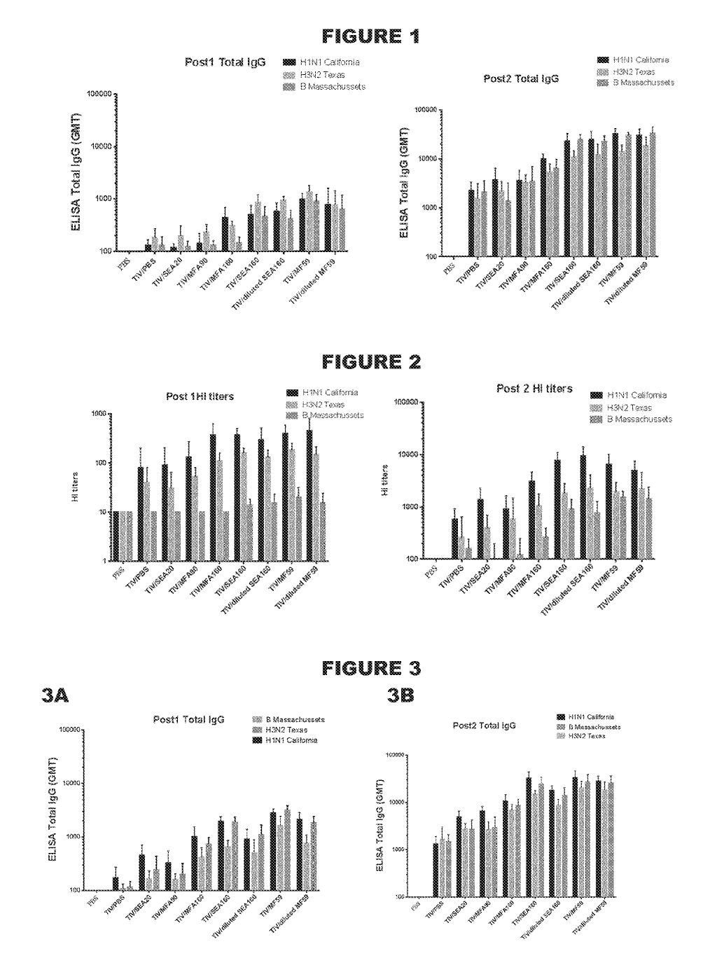

FIG. 2—ELISA results for 1 μg TIV adjuvanted study.

FIG. 3—Hemagglutination inhibition titers (HAI) for 0.1 μg TIV adjuvanted study.

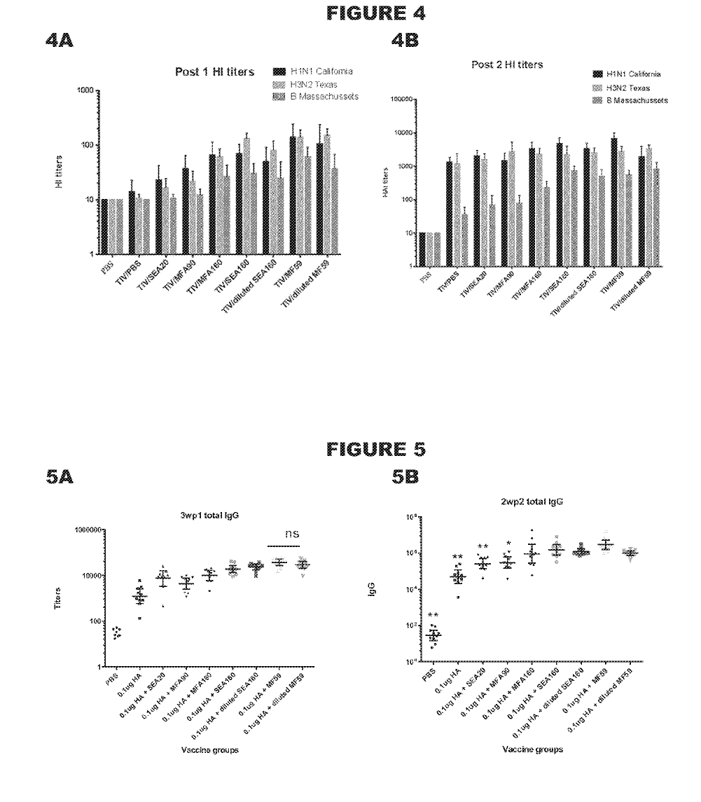

FIG. 4—Hemagglutination inhibition titers (HAI) for 1 μg TIV adjuvanted study.

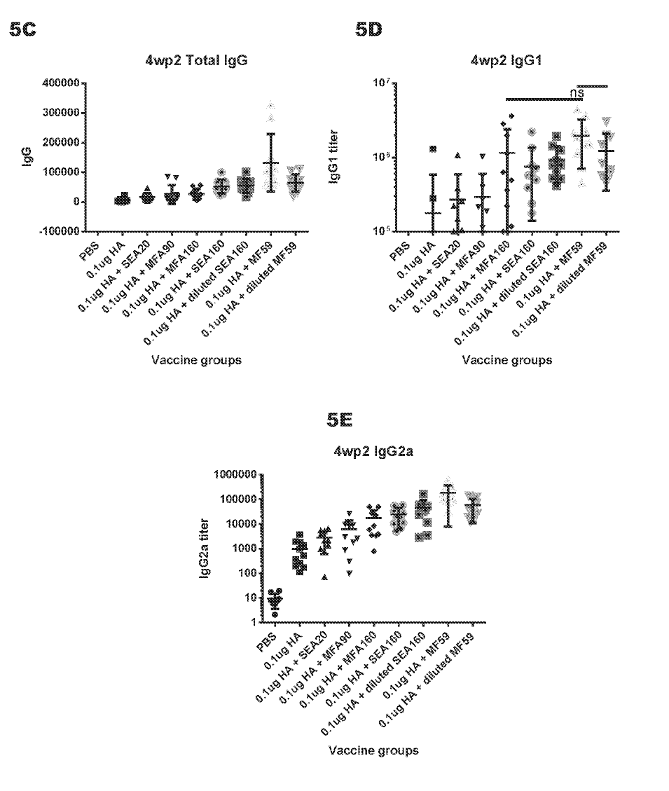

FIG. 5—ELISA results for 0.1 μg HA monobulk adjuvanted study.

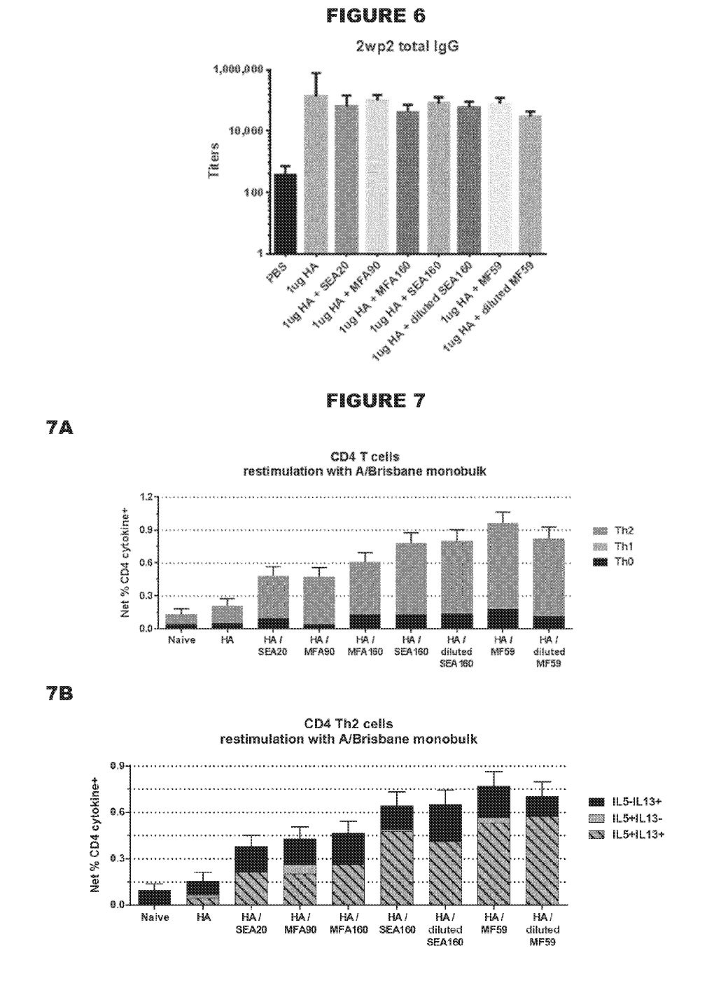

FIG. 6—ELISA results for 1 μg HA monobulk adjuvanted study.

FIG. 7—FACS of CD4+ positive re-stimulated with the protein used for immunization. 7A: the cells were sorted according to the type of response they generate: Th2, Th1 or Th0. 7B: within the Th2 population, the cells were further sorted for cells producing IL5, or IL13 or both.

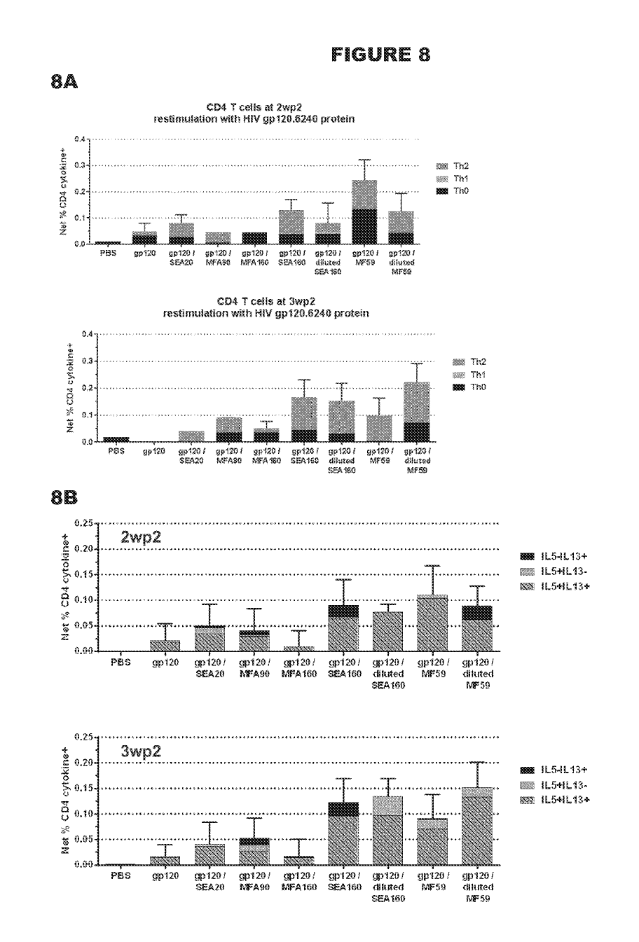

FIG. 8—FACS of CD4+ positive re-stimulated with the protein used for immunization. (A) The cells were sorted according to the type of response they generate: Th2, Th1 or Th0. (B) Within the Th2 population, the cells were further sorted for cells producing IL5, or IL13 or both.

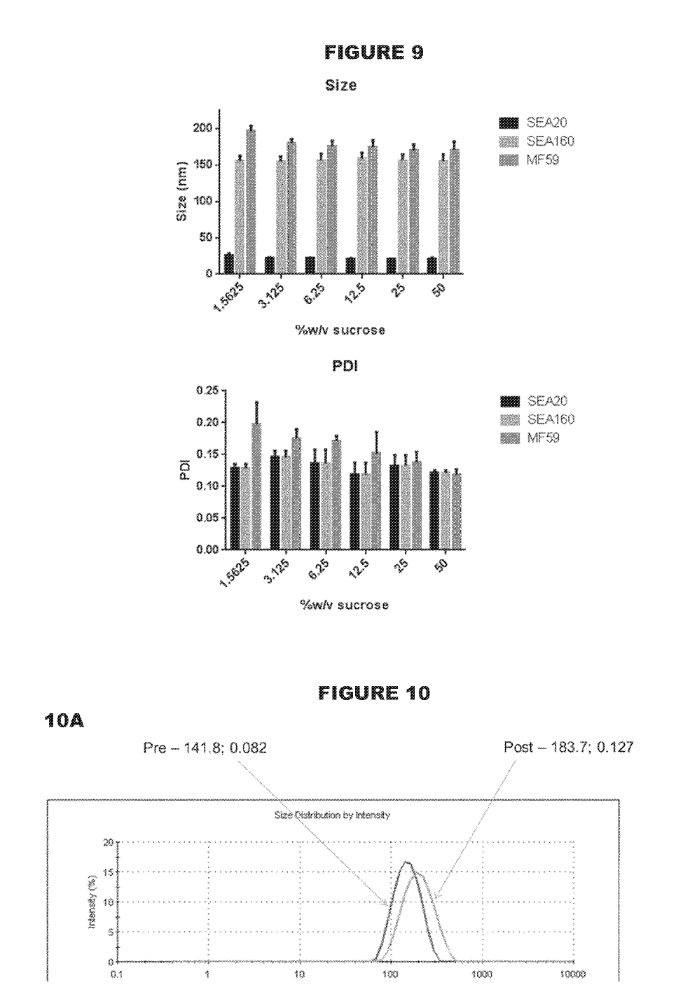

FIG. 9—Sizes and PDI post freeze thaw with different concentrations of sucrose and emulsion adjuvants. n=3 data expressed as average±standard deviation.

FIG. 10—Emulsion adjuvants mixed 1:1 with sucrose. The graph indicates the size (diameter, nm) pre- and post-lyophilization: (A) MF59 size and PDI pre lyophilization 141.8 nm and 0.082 respectively. Size post lyophilization 183.7 nm and 0.127 (B) SEA20 size and PDI pre-lyophilization 22.23 nm and 0.091 respectively. Size post-lyophilization 41.97 nm and 0.485. The blue peak is sucrose lyophilized and reconstituted (blank). (C) SEA160 size and PDI pre-lyophilization 147 nm and 0.083 respectively. Size post-lyophilization 179.6 nm and 0.104

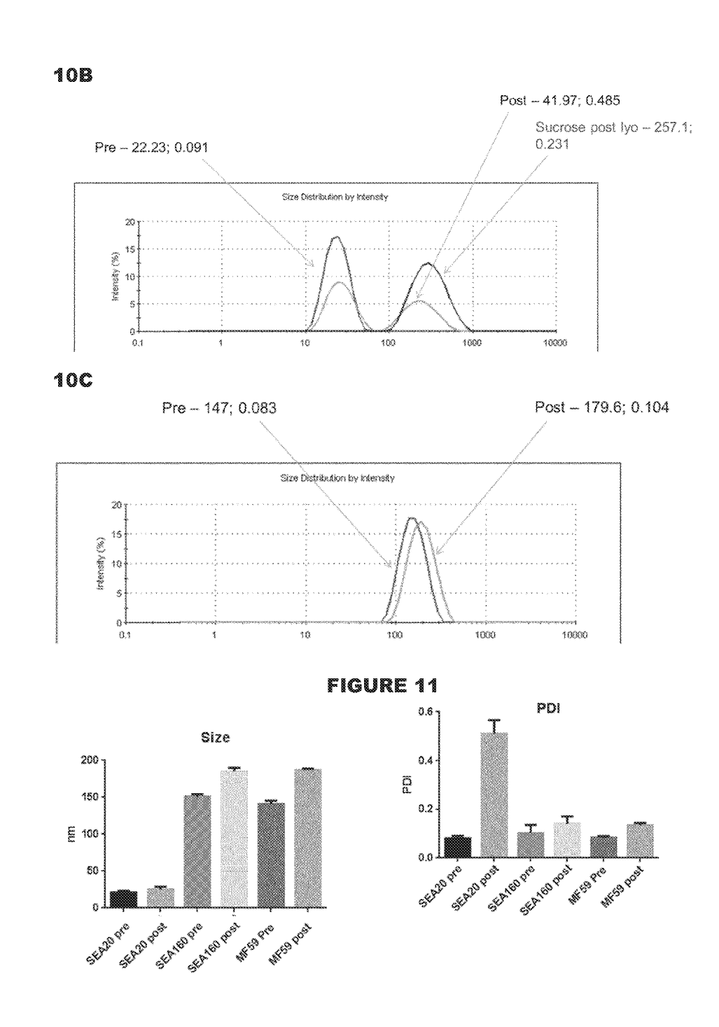

FIG. 11—n=3 results for emulsion adjuvants lyophilized with flu antigen. Sizes and PDI pre and post lyophilization, results expressed as mean±standard deviation.

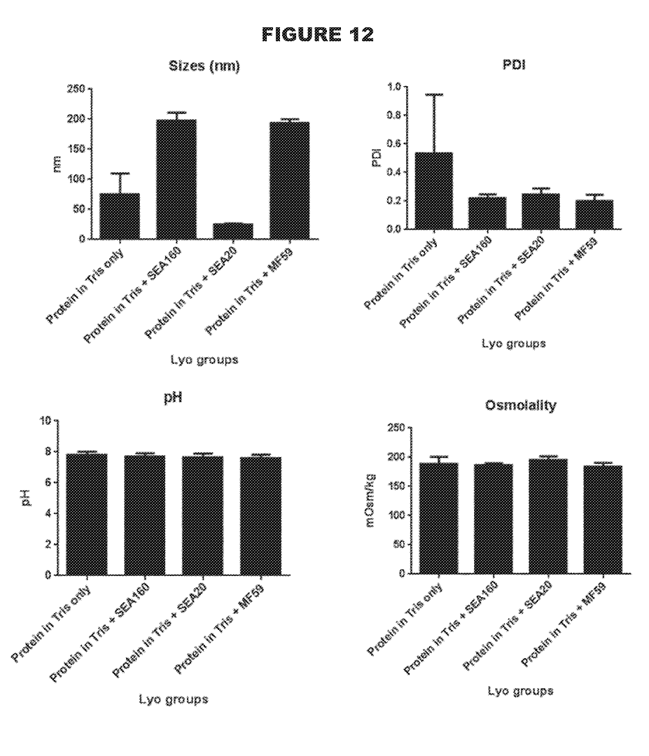

FIG. 12—Sizes, PDI, Osmolality and pH of reconstituted lyophilized vaccine groups. Results are representative for n=3.

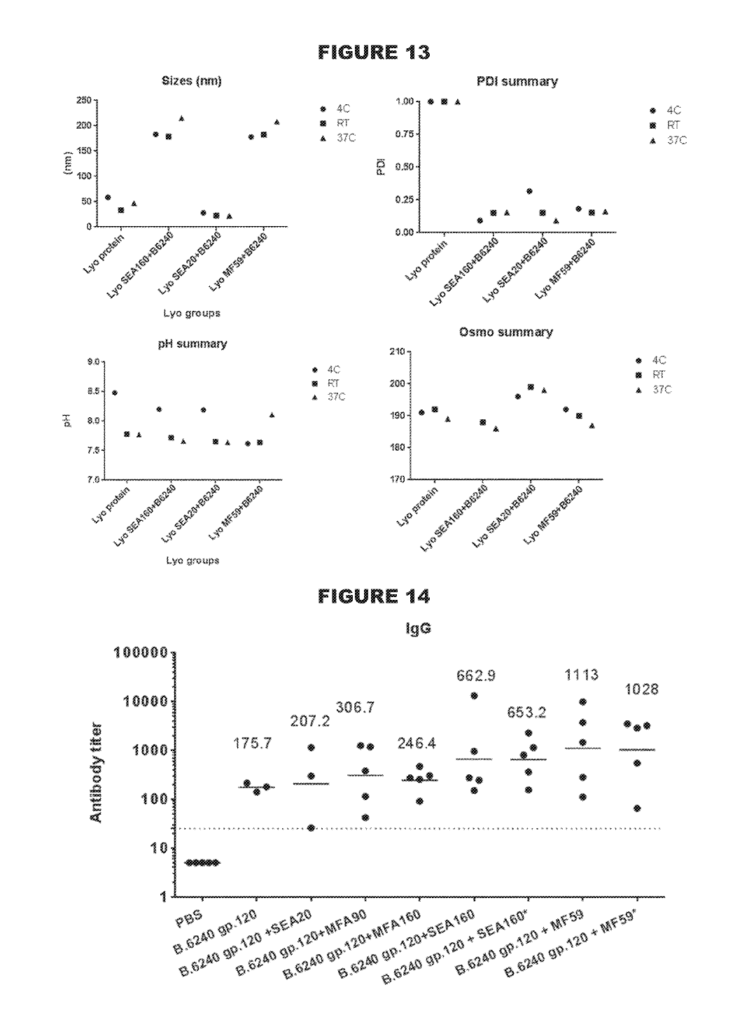

FIG. 13—Sizes, PDI, pH and Osmolality of formulations at T=10 days post lyophilization. For each group samples were stored at 4° C., RT or 37° C.

FIG. 14—ELISA titers measuring adjuvanted responses using HIV gp120 B.6240 vaccine antigen at 10 μg dose.

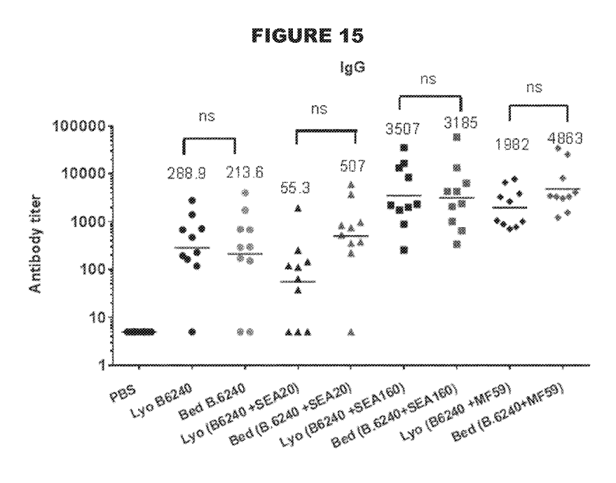

FIG. 15—IgG titers (2wp2) comparing the potency of single vial adjuvanted lyophilized formulations with their freshly mixed counterparts.

### EXAMPLE 1—PREPARATION OF EMULSIFYING MIXTURES

Mixtures of squalene, sorbitan trioleate and polysorbate 80 in various proportions were prepared. These were mixed at 37-40° C. overnight, and the next day were added to a 10-fold volume excess of citrate buffer (10 mM citrate, pH 6.5) at room temperature. The resulting emulsions were studied for average oil droplet size, PdI, and adjuvanticity.

Some of the self-emulsifying mixtures were able to produce emulsions having droplets as small as 20 nm with a PdI of only 0.08 (e.g. the emulsion referred to as ‘SEA20’, made using a mixture of 30% squalene, 10% sorbitan trioleate, and 70% polysorbate 80), but adjuvanticity did not match that of MF59. After evaluating the results, it was decided to seek a self-emulsifying mixture which could provide an emulsion with a diameter of around 160 nm with a relatively high squalene concentration.

Five systems with at least 50% squalene were tested, A-E:

TABLE 1

| | A | B | C | D | E |
|---|---|---|---|---|---|
| Squalene | 50 | 60 | 70 | 70 | 70 |
| Sorbitan trioleate | 10 | 10 | 10 | 15 | 20 |
| Polysorbate 80 | 40 | 30 | 20 | 15 | 10 |

Figures show % by volume

These were added to a 20-fold or 40-fold excess of 10 mM citrate buffer, pH 6.5, and the resulting emulsions had the following characteristics:

TABLE 2

| | | | | |
|---|---|---|---|---|
| A | B | C | D | E |

20-fold dilution

| | | | | | |
|---|---|---|---|---|---|
| Diameter (nm) | 66.1 | 89.5 | 84.5 | 188.0 | 2798 |
| PdI | 0.16 | 0.14 | 0.16 | 0.19 | 0.88 |

40-fold dilution

| | | | | | |
|---|---|---|---|---|---|
| Diameter (nm) | 62.4 | 89.8 | 85.4 | 182.9 | 318 |
| PdI | 0.13 | 0.14 | 0.16 | 0.14 | 0.62 |

Thus mixture ‘D’ met the target emulsion size, so this mixture was studied in more detail. Further experiments, performed at a controlled temperature, gave slightly smaller droplets (in the range of 150-160 nm) and lower PdI (<0.13). With a 20-fold dilution the emulsion has 3.5% by volume squalene, and 0.5% each of polysorbate 80 and sorbitan trioleate. For comparison, MF59 has 4.3% squalene and 0.75% of each surfactant (with a droplet size of around 150 nm, and a PdI of around 0.15). Unlike MF59, however, mixture ‘D’ (and also the other mixtures) can be prepared by simple manual mixing, without specialized equipment, and emulsification occurs spontaneously when the oil and surfactant components are added to water.

The emulsion formed from mixture ‘D’ at a 20-fold dilution, referred to as ‘SEA160’, was mixed with a monovalent influenza vaccine and assessed by SDS-PAGE. The antigen/emulsion mixture showed essentially the same protein bands as the antigen alone, and as an antigen/MF59 mixture, indicating that the emulsion is physicochemically compatible with protein antigens.

### EXAMPLE 2—EVALUATING THE ADJUVANT EFFECT OF NOVEL EMULSIONS AND STUDYING THE EFFECT OF DROPLET SIZE ON ADJUVANT RESPONSES IN VIVO USING MODEL ANTIGENS LIKE OVALBUMIN AND SUBUNIT INFLUENZA PROTEINS

Various adjuvants, including SEA160, were tested with trivalent inactivated influenza virus antigens (TIV) at Novartis Vaccines (now GSK Vaccines, Siena, Italy). Two studies were performed with different doses of following antigens: H1N1 A/California/7/09; H3N2 A/Texas/50/2012; and B/Massachusetts/2/2012. These antigens were tested at a 0.1 μg and a lag dose each in two different set of studies (Table 3 and Table 4 respectively). The antigens are standardized for hemagglutinin (HA) content by single-radial-immunodiffusion (SRID) as recommended by regulatory authorities. Adjuvants SEA160 and MF59 were additionally diluted for groups G and I to match squalene concentration in groups C, D and E.

TABLE 3
Study design of TIV antigens at 0.1 μg dose

| Group | Treatment | # of animals |
|---|---|---|
| A | 1x PBS | 10 |
| B | 0.1 μg TIV | 10 |
| C | 0.1 μg TIV + SEA20 | 10 |
| D | 0.1 μg TIV + MFA90 | 10 |
| E | 0.1 μg TIV + MFA160 | 10 |
| F | 0.1 μg TIV + SEA160 | 10 |
| G | 0.1 μg TIV + 3 times diluted SEA160 | 10 |
| H | 0.1 μg TIV + MF59 | 10 |
| I | 0.1 μg TIV + 4 times diluted MF59 | 10 |

TABLE 4
Study design of TIV antigens at 1 μg dose.

| Group | Treatment | # of animals |
|---|---|---|
| A | 1x PBS | 10 |
| B | 1 μg TIV | 10 |
| C | 1 μg TIV + SEA20 | 10 |
| D | 1 μg TIV + MFA90 | 10 |
| E | 1 μg TIV + MFA160 | 10 |
| F | 1 μg TIV + SEA160 | 10 |
| G | 1 μg TIV + 3 times diluted SEA160 | 10 |
| H | 1 μg TIV + MF59 | 10 |
| I | 1 μg TIV + 4 times diluted MF59 | 10 |

Two intramuscular immunizations were done three weeks apart. Animals were bled at the beginning of the study, 3wp1 and 2wp2. The sera were analyzed for IgG and hemagglutination inhibition titers. The HI titer is defined as the greatest serum dilution at which complete agglutination inhibition is observed. Results are expressed in FIGS. 1 to 4. ELISA titers and HAI titers indicate that 160 nm generate more potent responses than non-adjuvanted vaccines or lower sized emulsion adjuvants. Ordinary one way ANOVA and post hoc analysis by Dunnett's multiple comparisons indicate that SEA160 is statistically not different from MF59 for all antigens after second immunization (analysis not shown here). However, non-adjuvanted or lower sized adjuvants are significantly lower than MF59. HAI titers also indicate similar responses for SEA1160 and MF59.

A similar study was done at Cambridge using H1N1 monobulk antigen using the same two doses. The sera after two immunizations were analyzed for ELISA titers and a similar trend to the previous flu studies was observed for 0.1 μg antigen dose (FIG. 5). Additionally the 4wp2 sera were analyzed for IgG1 and IgG2a antibodies (FIG. 5). However, for this antigen at 1 μg dose we observed a saturation of the immune response in the non-adjuvanted group (FIG. 6). The animals from the 0.1 μg dose study were sacrificed at four weeks post second immunization (4wp2) and the spleens were harvested for T cell CFC assay (similar to the one done with model antigen ovalbumin). We observed a size dependent effect on T-cell responses with SEA160 and MF59 generating a similar profile (FIG. 7). We have compiled the results below where the vaccines exhibit a Th2 biased profile with larger adjuvant droplets being positive for both 1L5 and IL13 producing cells.

For the 0.1 μg study, one way ANOVA with post hoc by Dunnett's multiple comparison using MF59 as comparator for 3wp1 sera showed statistical difference with all groups expect diluted MF59 adjuvanted group. One way ANOVA with post hoc by Dunnett's multiple comparison using MF59 as comparator for 2wp2 sera showed statistically different result for naive (PBS), non-adjuvanted group and SEA20, MFA90 adjuvanted groups after the second immunization. One way ANOVA with post hoc by Dunnett's multiple comparison using MF59 as comparator for 4wp2 sera showed all groups to be statistically different. One way ANOVA with post hoc by Dunnett's multiple comparison using MF59 as comparator for 4wp2 sera for IgG1 analysis showed only diluted MF59 to be statistically not different. One way ANOVA with post hoc by Dunnett's multiple comparison using MF59 as comparator for 4wp2 sera for Ig2a analysis showed all groups to be statistically different.

For the 1 μg study, one way ANOVA with post hoc by Dunnett's multiple comparisons using MF59 as comparator showed no statistical difference with every group

### EXAMPLE 3—EVALUATING THE EFFECT OF DROPLET SIZE ON ADJUVANT RESPONSES IN VIVO USING HIV ENVELOPE ANTIGENS

The emulsion adjuvants used for TIV study were analyzed with HIV Env proteins to study the potency of these adjuvants with HIV antigens. Antigen used was HIV gp120 Thai protein B.6240 at 10 μg dose (Table 5). Antigen and adjuvant were mixed at a 1:1 ratio half hour prior to administration. We immunized female Balb/c twice, three weeks apart. Half of the animals were sacrificed at 2wp2 and the remaining at 3wp2. For both time-points spleens were harvested and a T cell CFC assay was done similar to the one done with model antigens ovalbumin and TIV (FIG. 8). We observe that the antigen gp120 gives a higher Th2 biased response when used with emulsion adjuvants—MF59, SEA160. Both MF59 and SEA160 with their diluted groups generate a higher Th2 response with major cells being positive for IL5 and IL13. This is similar to the trend we have observed with the flu antigen. The sera will now be analyzed by ELISA for IgG.

TABLE 5
(A) Study design and (B) Timeline for
the study of adjuvants with gp120.
A.

| | | # of |
|---|---|---|
| Group | Treatment | animals |
| A | 1X PBS | 10 |
| B | 10 μg HIV gp120 B.6240 | 10 |
| C | 10 μg HIV gp120 B.6240 + SEA20 | 10 |
| D | 10 μg HIV gp120 B.6240 + MFA90 | 10 |
| E | 10 μg HIV gp120 B.6240 + MFA160 | 10 |
| F | 10 μg HIV gp120 B.6240 + SEA160 | 10 |
| G | 10 μg HIV gp120 B.6240 + Diluted | 10 |
| | SEA160 | |
| H | 10 μg HIV gp120 B.6240 + MF59 | 10 |
| I | 10 μg HIV gp120 B.6240 + Diluted | 10 |
| | MF59 | |

B.

| Day | Procedure |
|---|---|
| 0 | Pre Bleed |
| 1 | 1^{st} IM |
| 20 | 3wp1 Bleed |
| 21 | 2^{nd} IM |
| 35 | 2wp2 Bleed and spleen harvest |
| | for 5 animals per group |
| 36 | FACS for 2wp2 |
| 42 | Terminal Bleed and spleen |
| | harvest for remaining 5 animals |
| 43 | FACS for 4wp2 |

Sera from these animals was analyzed by ELISA for total IgG at the 3wp2 time-point (see FIG. 14). One way ANOVA with post hoc analysis by Dunnett's multiple comparison tests showed no statistical difference between any groups. The numbers at the top of each group indicate their geometric mean titers. The titers clearly indicated the same trend as seen in the flu study. In comparison to non-adjuvanted group, SEA160 and MF59 had higher titers. The diluted groups of SEA160 and MF59 gave higher GMTs than the non-adjuvanted group. While SEA160 and diluted SEA160 had three fold higher titers than non-adjuvanted group, MF59 and diluted MF59 had almost six fold higher titers. However, the other three adjuvanted groups: SEA20, MFA90 and MFA160 did not show any different response than the non-adjuvanted group (see FIG. 14). The T-cell responses and IgG readouts re-assert the flu study conclusion: SEA160 due to its composition and droplet size gives similar responses to MF59 and is a potent adjuvant for viral antigens like flu and HIV.

### EXAMPLE 4—LYOPHILIZATION STUDIES

Lyophilization is the process of removing water from a frozen sample by sublimation and desorption under vacuum. Lyophilization enables storage and use of vaccines independent of the cold chain. Because lyophilization improves the thermal stability of vaccines, it permits efficient distribution of vaccines in the developing world. Storage and shipping becomes relatively easy as the bulky liquid vaccines formulations are transformed to dry cake-like forms. Lyophilization of protein, live-attenuated or inactivated virus, or bacteria-containing vaccines is a routine practice for prolonging shelf-life and increasing resistance to thermal stress. However, lyophilized adjuvanted vaccines have not been reported yet. Adjuvanted vaccines have added components that may create technical issues in successful lyophilization. Hence cold chain storage becomes crucial to retain the stability of different components—antigen and adjuvants (as in some cases antigen and adjuvants are mixed immediately prior to administration). If antigen and adjuvant can be lyophilized in a single vial, cold chain maintenance can be avoided and the mixing of adjuvant and antigen prior to administration can be replaced by the simpler process of reconstituting lyophilized vaccine with a diluent.

The first step to obtain a lyophilized formulation is to identify a good cryoprotectant for the vaccine formulation. Different cryoprotectants were mixed 1:1 with SEA20, frozen at −80° C. overnight and thawed the next day to analyze for size and PDI. Results are presented in Table 6. The original size of SEA20 after formulation was 21.66 nm and PDI of 0.062. These results clearly indicated that 6% sucrose in water gives similar result to the original size of SEA20 prior to freeze thaw without the cryoprotectant. Rest of the cyroprotectants either increases the size or PDI of SEA20 post freeze thaw.

TABLE 6
Screen of Cryoprotectants for Emulsion Adjuvants

| | | | | Size | PDI |
|---|---|---|---|---|---|
| | | | Thawed | after | after |
| Cryo | Cryo Name | Temp ° C. | mix | thaw (nm) | thaw |
| A | 6% Sucrose in Water | −80 | Clear | 22.39 | 0.058 |
| B | D-Trehalose dihydrate | −80 | Clear | 29.29 | 0.237 |
| | 6% in water | | | | |
| C | D-lactose monohydrate | −80 | Clear | 37.54 | 0.283 |
| | 6% in water | | | | |
| D | Maltose hydrate 6% in | −80 | Clear | 26.3 | 0.2 |
| | water | | | | |
| E | Raffinose pentahydrate | −80 | Clear | 35.62 | 0.338 |
| | 6% in water | | | | |
| F | Mannitol 6% in water | −80 | Clear | 42.82 | 0.355 |
| G | Fructose 6% in water | −80 | Clear | 22.04 | 0.064 |
| H | Sorbitol 6% in water | −80 | Clear | 31.17 | 0.226 |
| I | Glycerol 10% in water | −80 | Clear | 24.62 | 0.055 |
| J | PEG300 10% in water | −80 | Clear | 25.83 | 0.057 |
| K | PEG 4600 10% in water | −80 | Hazy | 154.7 | 0.167 |
| L | Glycine 6% in water | −80 | Turbid | 85.92 | 0.298 |
| M | PVA 87-90% hydrolyzed | −80 | Turbid | 143.5 | 0.13 |
| | 2% in water | | | | |

Next different concentrations of sucrose were assessed by the same freeze thaw process with SEA20, SEA160 and MF59. The experiment was repeated thrice and the data post freeze thaw is presented in FIG. 9. All concentrations of sucrose except 1.56% w/v maintain the size and PDI similar to the one prior to freeze thaw.

These sucrose concentrations were used for the initial couple of lyophilization cycles on Labconco lyophilizer with the adjuvants (no protein). The final lyophilized product was reconstituted using water for injection and the size and PDI were measured using DLS (Table 7). Size and PDI values in formulations 1 to 12 are acceptable sizes and PDI, whilst the ones for formulations 13 to 18 indicate that the formulation increased in size and/or PDI post lyophilization.

With the Labconco lyophilizer the lyophilized product had a glassy appearance. So, the adjuvants were lyophilized on the Virtis lyophiler where the primary and secondary drying temperatures can be controlled. The lyo cycle is described in Table 8. Historically the lyo cycle for the flu antigen was established using 5% w/v sucrose in the final reconstituted sample. As the initial proof of concept was to try and lyophilize emulsion adjuvants with flu antigen, the adjuvants were lyophilized with sucrose such that the final sucrose concentration on reconstitution would be 5% w/v. Results are presented in FIG. 10.

On the Virtis lyophilizer we could control the sizes and PDI of all emulsion adjuvants and also get a good quality of the lyo product. The increase in size and PDI for SEA20 was due to the sucrose present (data not shown).

TABLE 7
Lyophilization of emulsion adjuvants without antigen: sizes and PDI
post reconstitution with water for injection. Data is for n = 2 and
is presented as average.

| | | % w/v | | | | |
|---|---|---|---|---|---|---|
| | | sucrose | | | | |
| | | added | | | | |
| | | for 1:1 | Average | | Std. Dev | |
| Number | Formulation | mixing | Size (nm) | PDI | Size (nm) | PDI |
| 1 | SEA20 | 1.5625 | 21.89 | 0.06 | 0.14 | 0.01 |
| 2 | SEA20 | 3.125 | 21.78 | 0.1 | 0.36 | 0.01 |
| 3 | SEA20 | 6.25 | 22.07 | 0.1 | 0.55 | 0 |
| 4 | SEA20 | 12.5 | 21.425 | 0.1 | 1.02 | 0.01 |
| 5 | SEA20 | 25 | 20.695 | 0.14 | 2.34 | 0.01 |
| 6 | SEA20 | 50 | 21.125 | 0.22 | 3.68 | 0.07 |
| 7 | SEA160 | 1.5625 | 159.45 | 0.11 | 17.46 | 0.03 |
| 8 | SEA160 | 3.125 | 138.95 | 0.18 | 1.06 | 0.08 |
| 9 | SEA160 | 6.25 | 154.3 | 0.18 | 1.41 | 0 |
| 10 | SEA160 | 12.5 | 161.3 | 0.20 | 9.19 | 0.02 |
| 11 | SEA160 | 25 | 156.95 | 0.20 | 17.88 | 0.02 |
| 12 | SEA160 | 50 | 149.6 | 0.21 | 0.70 | 0.02 |
| 13 | MF59 | 1.5625 | 192.6 | 0.23 | 22.06 | 0.01 |
| 14 | MF59 | 3.125 | 169.9 | 0.25 | 5.94 | |
| 15 | MF59 | 6.25 | 265.7 | 0.4 | 23.19 | 0.06 |
| 16 | MF59 | 12.5 | 260.5 | 0.32 | 26.16 | 0.1 |
| 17 | MF59 | 25 | 251.35 | 0.31 | 6.71 | 0.08 |
| 18 | MF59 | 50 | 280.4 | 0.31 | 9.19 | 0.01 |

TABLE 8
Lyophilization cycle for emulsion adjuvants
with or without the antigen

| | | Time | Ramp/ | Vacuum |
|---|---|---|---|---|
| Step | Temp ° C. | (min) | Hold (R/H) | (mTorr) |
| Freezing | −50 | 240 | H | Door seal |
| Additional Hold | −50 | 15 | H | 2000 |
| Primary Drying | −34 | 90 | R | 200 |
| | −34 | 1680 | H | 10 |
| | −5 | 130 | R | 10 |
| | −5 | 600 | H | 10 |
| | 5 | 10 | R | 10 |
| | 5 | 600 | H | 10 |
| Secondary Drying | 8 | 1200 | H | 100 |
| Condenser Temp | −40 | | | |

Next, the adjuvants were lyophilized with H1N1 A/Brisbane monobulk antigen (flu antigen) in a single vial using the Virtis lyophilizer. So, the antigen and the adjuvant were mixed 1:1 such that the final vaccine dose contains 0.1 μg of antigen. While sucrose was prepared in deionized water, antigen was prepared using 2×PBS. Using the lyo cycle mentioned in table 12 the vaccines were lyophilized. Upon reconstitution size and PDI were measured using DLS and the antigen integrity was assessed with SDS-PAGE. Results are presented in FIG. 11.

Results indicate that sizes of adjuvants post reconstitution do not drastically increase. PDI indicates relative uniformity of the droplets. The increase in SEA20 is due to the presence of sucrose which can be subtracted by analyzing a lyophilized sucrose sample. The SDS-PAGE analysis indicates that the antigen integrity is maintained post lyophilization. The lyophilized samples in comparison to fresh non-adjuvanted flu antigen exhibited a similar band distribution (data not shown).

Once a single vial adjuvanted lyophilized formulation was possible, we tried to lyophilize HIV Env gp120 protein-B.6240. The protein was prepared in 20 mM Tris Buffer at pH between 7.5 to 8. Using the same lyophilization process, the antigens, adjuvants and sucrose were mixed such that the final reconstituted formulation contains 10 μg B.6240 and 5% w/v sucrose. This formulation is intended to be injected in to animals once optimized. So the final reconstituted samples were analyzed for size, PDI, pH, osmolality and antigen integrity by SDS-PAGE. Results are presented in FIG. 12.

Results from FIG. 12 indicate that the emulsion size and polydispersity do not increase post lyophilization. Additionally as the pH does not decrease during lyophilization, the protein is protected and does not undergo clipping. Based on the gel data we can effectively compare the antigen integrity of the lyophilized formulation with the frozen control. The less osmolality of the formulations will be optimized prior to injecting these in mice. Once it was established that a single vial lyophilized adjuvanted formulation is maintaining the adjuvant droplet size and protecting the protein from clipping, these samples were lyophilized and stored at 4° C., RT and 37° C. for 10 days to study their stability at higher temperatures. Results are presented in FIG. 13.

Based on these results, we observed that even at higher temperatures like 37° C. the samples exhibit similar results to those presented with fresh samples. Based on these results further experiments focussed on:
1. Increasing the osmolality of the formulations up to 270-330 mOsm/kg for in vivo use
2. Studying the protein integrity post lyophilization on Luminex by assaying the binding of the lyophilized protein to monoclonal antibodies and comparing it with fresh protein binding
   A further in vivo study was conducted to compare the potency of these lyophilized formulations with the freshly mixed formulations.

TABLE 9
In vivo study comparing the potency of single vial freshly
reconstituted adjuvanted lyophilized formulations with their
multi vial freshly mixed adjuvanted counterparts

| Group | Vaccine | Number of animals |
|---|---|---|
| A | PBS | 10 |
| B | Lyophilized B.6240 | 10 |
| C | Lyophilized (B.6240 + SEA20) | 10 |
| D | Lyophilized (B.6240 + SEA160) | 10 |
| E | Lyophilized (B.6240 + MF59) | 10 |
| F | Freshly prepared B.6240 | 10 |
| G | Freshly mixed (B.6240 + SEA20) | 10 |
| H | Freshly mixed (B.6240 + SEA160) | 10 |
| I | Freshly mixed (B.6240 + MF59) | 10 |

In this in vivo study two immunizations were performed three weeks apart and a total of 10 animals per group were used. Group A received PBS as it was the negative control. Groups B-E are single vial lyophilized groups containing B.6240, B.6240+SEA20, B.6240+SEA160 and B.6240+MF59. These were lyophilized prior to each IM and reconstituted with water for injection thirty minutes prior to administration. Groups F-I were mixed for immunizations thirty minutes prior to administration.

Sera from 2wp2 was analyzed for IgG titers and statistical tests were run between the lyophilized and freshly mixed groups (see FIG. 15). Three major inferences occurred from the results. Firstly, the antigen is very weakly immunogenic and even with adjuvants it does not give a higher boost (even with MF59). Using one-way ANOVA with post hoc analysis by Bonferroni's multiple tests, it was established that between the lyophilized and freshly mixed population there is no statistical difference, indicated by the “ns”. The numbers at the top of each group indicate the geometric mean titers. Although there is no statistical significance, the lyophilized SEA160 and MF59 adjuvanted formulations generate twelve and six fold higher titers than the lyophilized B.6240 respectively.

### EXAMPLE 5—DETERMINING THE MECHANISM OF ACTION OF THE NOVEL ADJUVANTS IN VITRO

From the in vivo studies assessing the cellular and humoral responses we determine the end-point effect of these adjuvants, but it is equally important to understand how this effect is generated. Studying the mechanism of action of adjuvants is difficult, but recently some literature around MF59 and alum has exhibited how interaction of injected adjuvants with innate immune system leads to a well-defined and specific immune response. Using “Vaccine adjuvants alum and MF59 induce rapid recruitment of neutrophils and monocytes that participate in antigen transport to draining lymph nodes” by Calabro, et al (Vaccine; 2011) as a reference we will try to study the immune cell recruitment at the site of injection (SOI) and antigen uptake and translocation to draining lymph nodes with and without novel emulsion adjuvants. We will use ovalbumin conjugated with AlexaFluor 647 (OVA-A647) non-adjuvanted and OVA-A647 adjuvanted with SEA20, MFA160 and SEA160. At 6 hr, 24 hr, 48 hr and 72 hr (Table 10) draining lymph nodes and quadriceps (site of injection) will be isolated and homogenized to form a single cell suspension. Using multi-color FACS we will study the immune cell recruitment at the quadriceps for antigen presenting cells, other immune cells like lymphocytes and OVA-A647 positive immune cells. Using the same scheme of FACS, we will study the antigen translocation in draining lymph nodes by studying the number of OVA-A647 positive immune cells at various time-points. The muscle and the lymph node data together will explain the difference in infiltration of immune cells at the site of injection, antigen uptake by immune cells and the migration of the antigen loaded immune cells to draining lymph nodes due to different emulsion adjuvants.

TABLE 10
Proposed study design for mechanistic
evaluation of novel emulsion adjuvants

| | | | Time-points | Tissues to |
|---|---|---|---|---|
| Group | Antigen | Adjuvant | to be assessed | be studied |
| 1 | — | — | Any | Both draining lymph |
| | | | | nodes and quadri- |
| | | | | ceps |
| 2 | OVA-A647 | — | 6 hr, 24 hr, | Both draining lymph |
| | | | 48 hr and 72 hr | nodes and quadri- |
| | | | | ceps |
| 3 | OVA-A647 | SEA20 | 6 hr, 24 hr, | Both draining lymph |
| | | | 48 hr and 72 hr | nodes and quadri- |
| | | | | ceps |
| 4 | OVA-A647 | MFA160 | 6 hr, 24 hr, | Both draining lymph |
| | | | 48 hr and 72 hr | nodes and quadri- |
| | | | | ceps |
| 5 | OVA-A647 | SEA160 | 6 hr, 24 hr, | Both draining lymph |
| | | | 48 hr and 72 hr | nodes and quadri- |
| | | | | ceps |

### EXAMPLE 6—DETERMINING THE BIO-DISTRIBUTION OF THESE NOVEL VACCINE PREPARATIONS

Adjuvanted vaccine preparations are formulated by mixing antigen and adjuvant prior to immunization. Bio-distribution studies with MF59 have demonstrated that the antigen and adjuvant have independent kinetics and clearance once administered intramuscularly. We will use orthogonal techniques like two-photon microscopy and in vivo imaging system (IVIS) to study the bio-distribution of fluorescently labeled emulsion adjuvants and fluorescently labeled ovalbumin from the site of injection. With two-photon imaging we will observe the intra-vital translocation of the labeled antigen and adjuvant to draining lymph nodes post immunization. Additionally we will monitor the relative loss in signal from the SOT during the translocation of antigen and adjuvant. With IVIS, we will monitor the overall bio-distribution of the emulsion adjuvants and antigen post immunization in live anesthetized animals. The major tissues of interest will be SOI and draining lymph nodes.

It will be understood that the invention has been described by way of example only and modifications may be made whilst remaining within the scope and spirit of the invention. The embodiments within the specification provide an illustration of embodiments of the invention and should not be construed to limit the scope of the invention. The skilled artisan readily recognizes that many other embodiments are encompassed.

### REFERENCES

[1] WO90/14837.
[2] Podda & Del Giudice (2003) Expert Rev Vaccines 2:197-203.
[3] Podda (2001) Vaccine 19: 2673-2680.
[4] Vaccine Design: The Subunit and Adjuvant Approach (eds. Powell & Newman) Plenum Press 1995 (ISBN 0-306-44867-X).
[5] Vaccine Adjuvants: Preparation Methods and Research Protocols (Volume 42 of Methods in Molecular Medicine series). ISBN: 1-59259-083-7. Ed. O'Hagan.
[6] New Generation Vaccines (eds. Levine et al.). 3rd edition, 2004. ISBN 0-8247-4071-8.
[7] O'Hagan (2007) Expert Rev Vaccines 6(5):699-710.
[8] Garçon et al. (2012) Expert Rev Vaccines 11:349-66.
[9] WO2006/100109.
[10] US2007/0014805.
[11] Light Scattering from Polymer Solutions and Nanoparticle Dispersions (W. Schartl), 2007. ISBN: 978-3-540-71950-2.
[12] WO2011/067669
[13] WO90/04609.
[14] Lidgate et al (1992) Pharmaceutical Research 9(7):860-863.
[15] Hoffmann et al. (2002) Vaccine 20:3165-3170.
[16] Subbarao et al. (2003) Virology 305:192-200.
[17] Liu et al. (2003) Virology 314:580-590.
[18] Ozaki et al. (2004) J. Virol. 78:1851-1857.
[19] Webby et al. (2004) Lancet 363:1099-1103.
[20] WO97/37000.
[21] Brands et al. (1999) Dev Biol Stand 98:93-100.
[22] Halperin et al. (2002) Vaccine 20:1240-7.
[23] Tree et al. (2001) Vaccine 19:3444-50.
[24] Kistner et al. (1998) Vaccine 16:960-8.
[25] Kistner et al. (1999) Dev Biol Stand 98:101-110.
[26] Bruhl et al. (2000) Vaccine 19:1149-58.
[27] Pau et al. (2001) Vaccine 19:2716-21.
[28] WO01/22992.
[29] Hehme et al. (2004) Virus Res. 103(1-2):163-71.
[30] Treanor et al. (1996) J infect Dis 173:1467-70.
[31] Keitel et al. (1996) Clin Diagn Lab Immunol 3:507-10.
[32] Williamson et al. (2006) Infection and Immunity 74: 961-7.
[33] Loukas et al. (2005) PLoS Med 2(10): e295.
[34] EP-A-0139417.
[35] Harper et al. (2004) Lancet 364(9447):1757-65.
[36] J Toxicol Clin Toxicol (2001) 39:85-100.
[37] Demicheli et al. (1998) Vaccine 16:880-884.
[38] Stepanov et al. (1996) J Biotechnol 44:155-160.
[39] WO2007/052155.
[40] WO2005/089837.
[41] U.S. Pat. No. 6,692,468.
[42] WO00/07647.
[43] WO99/17820.
[44] U.S. Pat. No. 5,971,953.
[45] U.S. Pat. No. 4,060,082.
[46] EP-A-0520618.
[47] WO98/01174.
[48] Banzhoff (2000) Immunology Letters 71:91-96.
[49] WO02/097072.
[50] Greenbaum et al. (2004) Vaccine 22:2566-77.
[51] Zurbriggen et al. (2003) Expert Rev Vaccines 2:295-304.
[52] Piascik (2003) J Am Pharm Assoc (Wash D.C.). 43:728-30.
[53] Mann et al. (2004) Vaccine 22:2425-9.
[54] Halperin et al. (1979) Am J Public Health 69:1247-50.
[55] Herbert et al. (1979) J Infect Dis 140:234-8.
[56] Chen et al. (2003) Vaccine 21:2830-6.

1. An oil/surfactant composition which, when mixed with an excess volume of surfactant-free aqueous material, can form an oil-in-water emulsion adjuvant, wherein said composition consists essentially of an oil component and a surfactant component, and wherein:
(i) the oil component makes up 51-85% by volume of the composition, and the composition can, when mixed with an excess volume of surfactant-free aqueous material, form an adjuvant having an average oil particle diameter of less than 220 nm;
(ii) the oil component makes up more than 50% by volume of the composition, the surfactant component consists of substantially equal volumes of two surfactants, and the composition can, when mixed with an excess volume of surfactant-free aqueous material, form an adjuvant having an average oil particle diameter of less than 220 nm;
(iii) the oil component makes up more than 50% by volume of the composition, the surfactant component has a HLB between 8 and 10, and the composition can, when mixed with an excess volume of surfactant-free aqueous material, form an adjuvant having an average oil particle diameter of less than 220 nm;
(iv) the oil component makes up more than 50% by volume of the composition, and the composition can, when mixed with an excess volume of surfactant-free aqueous material, form an adjuvant having an average oil particle diameter within the range of 140-200 nm;
or
(v) the oil component makes up more than 50% by volume of the composition, and the composition can, when mixed with an excess volume of surfactant-free aqueous material, form an adjuvant having an average oil particle diameter within the range of 140-175 nm.

2. The composition of claim 1, wherein the oil component makes up no more than 75% by volume e.g. wherein the oil component makes up 65-75% by volume.

3. The composition of claim 1, wherein the oil component comprises squalene.

4. The composition of claim 1, wherein the surfactant component is a mixture of two surfactants, which may be present at equal volumes.

5. The composition of claim 4, wherein the two surfactants comprise sorbitan trioleate and/or polysorbate 80.

6. The composition of claim 1, consisting essentially of squalene, sorbitan trioleate and polysorbate 80.

7. A method of forming an oil-in-water emulsion having an average oil particle diameter of less than 220 nm and comprising an oil component, an aqueous component, and a surfactant component, said method comprising: (i) providing an oil/surfactant composition according to claim 1; (ii) providing an aqueous component; (iii) combining the oil/surfactant composition with a volume excess of the aqueous component, to form a diluted composition; and (iv) gently mixing the diluted composition to form the oil-in-water emulsion.

8. The method of claim 7 wherein the oil-in-water emulsion has an average oil particle diameter of between 85-220 nm.

9. The method of claim 7, wherein the aqueous component includes a pH buffer e.g. buffered between pH 6.0 and pH 8.0.

10. The method of claim 7, further comprising a step of filter sterilising the oil-in-water emulsion.

11. The method of claim 7, further comprising a step of drying the emulsion.

12. An oil-in-water emulsion obtainable by the method of claim 7.

13. The emulsion of claim 12, having a polydispersity index of less than 0.15.

14. A lyophilisate of the oil-in-water emulsion of claim 13.

15. A kit comprising:
(I) an immunogen component; and the oil-in-water emulsion of claim 12; or
(II) the oil-in-water emulsion of claim 12 wherein the emulsion includes an immunogen.
